(19) United States
(12) Patent Application Publication
(10) Pub. No.: US 2026/0224358 A1
(43) Pub. Date: Aug. 6, 2026

(54) LEAFLETS FOR A PROSTHETIC HEART VALVE

(71) Applicant: Edwards Lifesciences Corporation, Irvine, CA (US)

(72) Inventors: Tamir S. Levi, Zikhron Yaakov (IL); Roy Shitrit, Zichron Yaakov (IL); Michael Bukin, Pardes Hanna (IL); Anatoly Dvorsky, Haifa (IL); Nikolai Gurovich, Hadera (IL)

(73) Assignee: Edwards Lifesciences Corporation, Irvine, CA (US)

(21) Appl. No.: 19/564,841

(22) Filed: Mar. 12, 2026

Related U.S. Application Data
(63) Continuation of application No. PCT/US2024/049206, filed on Sep. 30, 2024.
(60) Provisional application No. 63/587,399, filed on Oct. 2, 2023.

(51) Int. Cl. A61F 2/24

(52) U.S. Cl. CPC A61F 2/2418; A61F 2230/0006; A61F 2230/0026

(57) ABSTRACT

Leaflets for prosthetic heart valves are disclosed. As an example, a prosthetic heart valve comprises a frame and a plurality of leaflets mounted on an inside the frame, where each leaflet comprises a main body with a cusp edge portion and an outflow edge portion. Each leaflet is compressed in an X direction and/or Y direction to form a concave shape.

### CROSS-REFERENCE TO RELATED APPLICATIONS

This application is a continuation of PCT Application No. PCT/US2024/049206, filed Sep. 30, 2024, which claims the benefit of U.S. Provisional Patent Application No. 63/587,399 , filed Oct. 2, 2023, the entire contents of each of which are incorporated by reference herein.

### FIELD

The present disclosure relates to prosthetic heart valves, including leaflets for prosthetic heart valves.

### BACKGROUND

The human heart can suffer from various valvular diseases. These valvular diseases can result in significant malfunctioning of the heart and ultimately require repair of the native valve or replacement of the native valve with an artificial valve. There are a number of known repair devices (e.g., stents) and artificial valves, as well as a number of known methods of implanting these devices and valves in humans. Percutaneous and minimally-invasive surgical approaches are used in various procedures to deliver prosthetic medical devices to locations inside the body that are not readily accessible by surgery or where access without surgery is desirable. In one specific example, a prosthetic heart valve can be mounted in a crimped state on the distal end of a delivery apparatus and advanced through the patient's vasculature (e.g., through a femoral artery and the aorta) until the prosthetic valve reaches the implantation site in the heart. The prosthetic valve is then expanded to its functional size, for example, by inflating a balloon on which the prosthetic valve is mounted, actuating a mechanical actuator that applies an expansion force to the prosthetic valve, or by deploying the prosthetic valve from a sheath of the delivery apparatus so that the prosthetic valve can self-expand to its functional size.

Most expandable prosthetic heart valves comprise a radially expandable and compressible cylindrical metal frame or stent and prosthetic leaflets mounted inside the frame. Each leaflet can comprise a main body with a cusp edge portion and a set of commissure tabs extending from the main body on opposite sides of the leaflet. The leaflets can be secured to one another at adjacent commissure tabs to form commissures that are then secured to the frame of the prosthetic heart valve. The cusp edge portion of each leaflet can also be coupled to struts of the frame (either directly or through an inner skirt disposed around an inner surface of the frame). The leaflets of the prosthetic heart valve are configured to open and close to regulate a flow of blood through the prosthetic heart valve, from an inflow end to an outflow end of the prosthetic heart valve.

### SUMMARY

Described herein are prosthetic heart valves, delivery apparatuses, and methods for implanting prosthetic heart valves. Also described herein are leaflets configured to be mounted inside a frame of a prosthetic heart valve, methods for forming the leaflets, and methods for assembling and attaching the leaflets to the frame. The disclosed leaflets, prosthetic heart valves, and methods can, for example, provide leaflets with a more three-dimensional (3D) shape, such as increased concavity/convexity, that results in improved mobility and coaptation of the leaflets (e.g., the leaflets may fully coapt or close together, such that they contact one another at a level of the free or outflow edges of the leaflets). The methods and assemblies described herein can also provide prosthetic heart valves with decreased tissue overgrowth along the inflow end portions of the leaflets. As such, the devices, assemblies, and methods disclosed herein can, among other things, overcome one or more of the deficiencies of typical prosthetic heart valves.

A leaflet for a prosthetic valve can comprise a main body with an outflow edge portion and a cusp edge portion.

In some examples, the leaflet can comprise two tabs disposed on opposite sides of the main body.

In some examples, the cusp edge portion has a trapezoidal shape with two angled side edges and an inflow edge extending between the two angled side edges, wherein the inflow edge defines an inflow end of the cusp edge portion.

In some examples, the inflow edge extends in a circumferential direction relative to the frame.

In some examples, the inflow edge is perpendicular to a central longitudinal axis of the leaflet.

In some examples, the inflow edge is parallel to straight portions of the outflow edge portion.

In some examples, the outflow edge portion comprises a central bump.

In some examples, the outflow edge portion comprises two depressed regions that are depressed inward into the main body relative to the central bump, and the central bump is disposed between the two depressed regions.

In some examples, the leaflet is a three-dimensional leaflet where the main body has a concave shape when attached to an annular frame of a prosthetic heart valve.

In some examples, a portion of the cusp edge portion has a shape of a first cylinder intersecting with a second cylinder and the main body has a concave shape when attached to an annular frame of a prosthetic heart valve.

In some examples, the cusp edge portion has a curved shape with an angle of curvature in a range of 100 to 140 degrees.

In some examples, a leaflet comprises a main body with a cusp edge portion and an outflow edge portion. The leaflet further comprises two tabs disposed on opposite sides of the main body, adjacent to the outflow edge portion. The outflow edge portion comprises a central bump and two depressed regions that are depressed inward into the main body relative to the central bump, and the central bump is disposed between the two depressed regions.

In some examples, a three-dimensional leaflet comprises a main body with a cusp edge portion and an outflow edge portion, wherein a portion of the cusp edge portion has a shape of a first cylinder intersecting with a second cylinder and the main body has a concave shape when attached to an annular frame of a prosthetic heart valve.

In some examples, a leaflet comprises one or more of the components recited in Examples 58-91 and 101-105 below.

A prosthetic heart valve can comprise a frame and a valvular structure coupled to the frame. In addition to these components, a prosthetic heart valve can further comprise one or more of the components disclosed herein.

In some examples, a prosthetic heart valve can comprise a sealing member configured to reduce paravalvular leakage.

In some examples, the valvular structure comprises a plurality of leaflets mounted on an inside of the frame.

In some examples, each leaflet can comprise a main body with an outflow edge portion and a cusp edge portion.

In some examples, the leaflet can comprise two tabs disposed on opposite sides of the main body.

In some examples, the cusp edge portion of the leaflet has a trapezoidal shape with two angled side edges and an inflow edge extending between the two angled side edges, where the inflow edge defines an inflow end of the cusp edge portion.

In some examples, the inflow edge extends in a circumferential direction relative to the frame.

In some examples, the inflow edge is perpendicular to a central longitudinal axis of the leaflet.

In some examples, the inflow edge is parallel to straight portions of the outflow edge portion.

In some examples, the outflow edge portion comprises a central bump.

In some examples, the outflow edge portion comprises two depressed regions that are depressed inward into the main body relative to the central bump, and the central bump is disposed between the two depressed regions.

In some examples, the leaflet is a three-dimensional leaflet where the main body has a concave shape when attached to an annular frame of a prosthetic heart valve.

In some examples, a portion of the cusp edge portion has a shape of a first cylinder intersecting with a second cylinder and the main body has a concave shape when attached to an annular frame of a prosthetic heart valve.

In some examples, the cusp edge portion has a curved shape with an angle of curvature in a range of 100 to 140 degrees.

In some examples, the cusp edge portion is coupled to the frame such that a width of the leaflet measured from one side edge of the two angled side edges to the other side edge of the two angled side edges is smaller than a width of the leaflet prior to being coupled to the frame.

In some examples, the cusp edge portion is coupled to the frame such that the leaflet is compressed axially and/or laterally to form a concave shape.

In some examples, a prosthetic heart valve comprises a frame that is radially expandable from a radially collapsed configuration to a radially expanded configuration, where the frame has an inflow end and an outflow end. The prosthetic heart valve further comprises a plurality of leaflets mounted on an inside the frame, where each leaflet comprises a main body with a cusp edge portion and an outflow edge portion, where the cusp edge portion has a trapezoidal shape with two angled side edges and an inflow edge extending between the two angled side edges, and wherein the inflow edge defines an inflow end of the cusp edge portion which extends in a circumferential direction relative to the frame. The two angled side edges are coupled to the frame such that a material of the main body of the leaflet is compressed and forms a concave shape.

In some examples, a prosthetic heart valve comprises a frame that is radially expandable from a radially collapsed configuration to a radially expanded configuration, wherein the frame has an inflow end and an outflow end. The prosthetic heart valve further comprises a plurality of leaflets mounted on an inside the frame, where each leaflet comprises a main body with a cusp edge portion and an outflow edge portion, where the cusp edge portion has a trapezoidal shape with two angled side edges and an inflow edge extending between the two angled side edges, and where the inflow edge defines an inflow end of the cusp edge portion which extends in a circumferential direction relative to the frame. The cusp edge portion is coupled to the frame such that a width of the leaflet measured from one side edge of the two angled side edges to the other side edge of the two angled side edges is smaller than a width of the leaflet prior to being coupled to the frame.

In some examples, a prosthetic heart valve comprises a frame that is radially expandable from a radially collapsed configuration to a radially expanded configuration, where the frame has an inflow end and an outflow end. The prosthetic heart valve further comprises a plurality of leaflets mounted on an inside the frame, where each leaflet comprises a main body with a cusp edge portion and an outflow edge portion, where the cusp edge portion has a trapezoidal shape with two angled side edges and an inflow edge extending between the two angled side edges, and where the inflow edge defines an inflow end of the cusp edge portion which extends in a circumferential direction relative to the frame. The cusp edge portion is coupled to the frame such that the leaflet is compressed axially and/or laterally to form a concave shape.

In some examples, a prosthetic heart valve comprises one or more of the components recited in Examples 1-46, 106-111, and 113 below.

In some examples, a method can comprise coupling a leaflet to a frame of a prosthetic valve by moving two angled side edges of a cusp edge portion of the leaflet closer together to decrease a width of the leaflet that is measured between the two angled side edges and cause outward bulging of a main body of the leaflet and coupling the two angled side edges to the frame.

In some examples, the cusp edge portion of the leaflet has an inflow edge disposed between the two angled side edges and defining an inflow end of the cusp edge portion.

In some examples, a method of forming a three-dimensional leaflet can comprise cutting and intersecting a first cylinder representing a leaflet with a second cylinder to form a 3D cut leaflet shape and flattening the 3D cut leaflet shape into a 2D cut leaflet shape to form a 2D leaflet cutting template.

In some examples, the method can comprise cutting a leaflet material in a flattened configuration with the 2D leaflet cutting template to form a 3D leaflet with a rounded cusp edge.

In some examples, the method can comprise coupling the 3D leaflet to an annular frame of a prosthetic valve such that it assumes a 3D configuration where a main body of the 3D leaflet has a concave shape.

In some examples, a method of assembling a leaflet to a frame of a prosthetic valve comprises coupling the leaflet to the frame of the prosthetic valve, where the leaflet comprises a main body with a cusp edge portion and an outflow edge portion, where the cusp edge portion has two angled side edges and an inflow edge disposed between the two angled side edges and defining an inflow end of the cusp edge portion. Coupling the leaflet to the frame comprises moving the two angled side edges closer together to decrease a width of the leaflet that is measured between the two angled side edges and cause outward bulging of the main body and coupling the two angled side edges to the frame.

In some examples, a method of forming a three-dimensional (3D) leaflet for a prosthetic valve comprises cutting and intersecting a first cylinder representing a leaflet with a second cylinder to form a 3D cut leaflet shape and flattening the 3D cut leaflet shape into a 2D cut leaflet shape to form a 2D leaflet cutting template. The method further comprises cutting a leaflet material in a flattened configuration with the 2D leaflet cutting template to form a cut, 3D leaflet with a rounded cusp edge. The method further comprises coupling the 3D leaflet to an annular frame of the prosthetic valve such that it assumes a 3D configuration where a main body of the 3D leaflet has a concave shape.

In some examples, a method comprises one or more of the features recited in Examples 47-57, 92-100, and 112 below.

The various innovations of this disclosure can be used in combination or separately. This summary is provided to introduce a selection of concepts in a simplified form that are further described below in the detailed description. This summary is not intended to identify key features or essential features of the claimed subject matter, nor is it intended to be used to limit the scope of the claimed subject matter. The foregoing and other objects, features, and advantages of the disclosure will become more apparent from the following detailed description, claims, and accompanying figures.

### BRIEF DESCRIPTION OF THE DRAWINGS

FIG. 1 is a side view of a prosthetic heart valve, according to one example.

FIG. 2 is a side view of an example of a delivery apparatus configured to deliver and implant a radially expandable prosthetic heart valve at an implantation site.

FIG. 3 is a perspective view of a prosthetic heart valve, according to an example.

FIG. 4 is a schematic of exemplary leaflets having various trapezoidal-shaped cusp edges, where the exemplary leaflets are shown superimposed over a leaflet with a more traditional, curved cusp edge shape and a portion of a frame for a prosthetic heart valve.

FIG. 5A is a schematic of an exemplary leaflet with a trapezoidal-shaped cusp edge portion shown in its as-cut and off-frame configuration, superimposed over a leaflet with a more traditional, curved cusp edge shape.

FIG. 5B is a schematic depicting the leaflet of FIG. 5A in its compressed or on-frame configuration that results from moving the edges of the cusp edge portion toward one another when attaching to a prosthetic valve frame.

FIG. 6A is a schematic of an exemplary leaflet with a trapezoidal-shaped cusp edge portion shown in its as-cut and off-frame configuration, superimposed over a leaflet with a more traditional, curved cusp edge shape, and where the inflow edge of the cusp edge portion of the exemplary leaflet extends beyond an inflow edge of the traditional leaflet.

FIG. 6B is a schematic depicting the leaflet of FIG. 6A in its compressed or on-frame configuration that results from moving the edges of the cusp edge portion toward one another when attaching to a prosthetic valve frame.

FIG. 7A is a schematic of an exemplary leaflet with a trapezoidal-shaped cusp edge portion shown in its as-cut and off-frame configuration, superimposed over a leaflet with a more traditional, curved cusp edge shape, and where the inflow edge of the cusp edge portion of the exemplary leaflet is offset from an inflow edge of the traditional leaflet toward its outflow edge portion.

FIG. 7B is a schematic depicting the leaflet of FIG. 7A in its compressed or on-frame configuration that results from moving the edges of the cusp edge portion toward one another when attaching to a prosthetic valve frame.

FIG. 8A is a schematic of an exemplary leaflet with a trapezoidal-shaped cusp edge portion shown in its as-cut and off-frame configuration, superimposed over a leaflet with a more traditional, curved cusp edge shape, and where the inflow edge of the cusp edge portion of the exemplary leaflet extends beyond an inflow edge of the traditional leaflet and has a greater length that spaces side edges of the cusp edge portion farther away from one another.

FIG. 8B is a schematic depicting the leaflet of FIG. 8A in its compressed or on-frame configuration that results from moving the edges of the cusp edge portion toward one another when attaching to a prosthetic valve frame.

FIG. 9A is a schematic of an exemplary leaflet with a trapezoidal-shaped cusp edge portion shown in its as-cut and off-frame configuration, superimposed over a leaflet with a more traditional, curved cusp edge shape, and where the inflow edge of the cusp edge portion of the exemplary leaflet extends has a greater length that spaces side edges of the cusp edge portion farther away from one another, and where commissure tabs of the exemplary leaflet are angled and non-parallel relative to a central longitudinal axis of the leaflet.

FIG. 9B is a schematic depicting the leaflet of FIG. 9A in its compressed or on-frame configuration that results from moving the edges of the cusp edge portion toward one another when attaching to a prosthetic valve frame.

FIG. 10 shows the leaflet of FIG. 6A in its as-cut and off-frame configuration with dashed lines across portions of the leaflet showing the relative positioning of the portions of the leaflet when attached to the frame of the prosthetic valve of FIG. 11.

FIG. 11 shows the leaflet of FIG. 10 attached to the frame of the prosthetic valve with the inflow edge of the cusp edge portion of the leaflet offset from the inflow end of the frame and the side edges of the cusp edge portion of the leaflet coupled to the frame, which results in compression and/or bulging of the material of the leaflet that creates a more 3D or concave shape of the leaflet when attached to the frame.

FIG. 12 shows an exemplary leaflet in a flattened configuration, where the leaflet comprises an outflow edge portion with a central protrusion or bump configured to improve coaptation between leaflets of a prosthetic heart valve.

FIG. 13 is a schematic top view of a prosthetic valve comprising the leaflets of FIG. 12 in a closed position of the valve.

FIG. 14 is a schematic top view of a prosthetic valve comprising leaflets without a central protrusion along their outflow edge portion, and the valve is shown in a closed position with a gap between the closed leaflets due to the leaflets not fully closing together along their cusp edge portions.

FIG. 15A shows an exemplary leaflet in a flattened configuration, where the leaflet comprises an outflow edge portion with two depressed regions and a central bump disposed between the two depressed regions, and where the central bump is axially aligned with outflow edges of the commissure tabs.

FIG. 15B shows the leaflet of FIG. 15B with the central bump axially offset away from outflow edges of the commissure tabs.

FIG. 16 shows an exemplary leaflet in a flattened configuration, where the leaflet comprises an outflow edge portion with two depressed regions and a central bump disposed between the two depressed regions and opposing commissure tabs that are angled away from the outflow edge portion.

FIG. 17 shows an exemplary leaflet in a flattened configuration, where the leaflet comprises an outflow edge portion with two depressed regions and a central bump disposed between the two depressed regions and opposing commissure tabs that are angled toward one another and the outflow edge portion.

FIG. 18 shows an exemplary leaflet with a rounded or circular cusp edge.

FIG. 19A is a front view of a first cylinder intersecting with a second cylinder at a perpendicular angle to form a 3D cut leaflet shape.

FIG. 19B is perspective view of the first cylinder intersecting with a second cylinder of FIG. 19A.

FIG. 19C is a side view of the first cylinder intersecting with a second cylinder of FIG. 19A.

FIG. 20 is a schematic of the 3D cut leaflet shape resulting from the method shown in FIGS. 19A-19C, and a 2D cut leaflet shape formed by flattening the 3D cut leaflet shape, where the 2D cut leaflet shape can be used to derive a two-dimensional cutting pattern or template used to cut a leaflet material in a flattened configuration and form a leaflet for a prosthetic valve.

FIG. 21 is a side view of a first cylinder intersecting with a second cylinder at a non-perpendicular angle to form a 3D cut leaflet shape.

FIG. 22 is a schematic of the 2D cut leaflet shape formed by the perpendicular intersecting cylinders of FIGS. 19A-19B superimposed over the 2D cut leaflet shape formed by the non-perpendicular intersecting cylinders of FIG. 21.

FIG. 23 is a side view of a prosthetic heart valve including a frame and a 3D leaflet, formed by cutting leaflet material with the 2D cut leaflet shape of FIG. 20, mounted inside the frame.

FIG. 24A is a side view of a prosthetic heart valve including a frame and a 3D leaflet, formed by cutting leaflet material with the longer 2D cut leaflet shape of FIG. 22, mounted inside the frame.

FIG. 24B is a perspective view of the prosthetic heart valve of FIG. 24A.

FIG. 25A shows a method for forming a leaflet with a rounded cusp edge by passing a perpendicularly extending cutting cylinder through an existing (previously cut) leaflet with a non-rounded cusp edge, while the leaflet is held in a flattened configuration.

FIG. 25B is a view of a portion of the leaflet resulting from the cutting in FIG. 25A.

FIG. 26A shows a method for forming a leaflet with a rounded cusp edge by passing a perpendicularly extending cutting cylinder through an existing (previously cut) leaflet with a non-rounded cusp edge, while the leaflet is held in a 3D configuration.

FIG. 26B is a view of a portion of the leaflet resulting from the cutting in FIG. 26A.

### DETAILED DESCRIPTION

### General Considerations

For purposes of this description, certain aspects, advantages, and novel features of examples of this disclosure are described herein. The disclosed methods, apparatus, and systems should not be construed as being limiting in any way. Instead, the present disclosure is directed toward all novel and nonobvious features and aspects of the various disclosed examples, alone and in various combinations and sub-combinations with one another. The methods, apparatus, and systems are not limited to any specific aspect or feature or combination thereof, nor do the disclosed examples require that any one or more specific advantages be present or problems be solved.

Although the operations of some of the disclosed examples are described in a particular, sequential order for convenient presentation, it should be understood that this manner of description encompasses rearrangement, unless a particular ordering is required by specific language set forth below. For example, operations described sequentially may in some cases be rearranged or performed concurrently. Moreover, for the sake of simplicity, the attached figures may not show the various ways in which the disclosed methods can be used in conjunction with other methods. Additionally, the description sometimes uses terms like “provide” or “achieve” to describe the disclosed methods. These terms are high-level abstractions of the actual operations that are performed. The actual operations that correspond to these terms may vary depending on the particular implementation and are readily discernible by one of ordinary skill in the art.

As used in this application and in the claims, the singular forms “a,” “an,” and “the” include the plural forms unless the context clearly dictates otherwise. Additionally, the term “includes” means “comprises.” Further, the term “coupled” generally means physically, mechanically, chemically, magnetically, and/or electrically coupled or linked and does not exclude the presence of intermediate elements between the coupled or associated items absent specific contrary language.

As used herein, the term “proximal” refers to a position, direction, or portion of a device that is closer to the user and further away from the implantation site. As used herein, the term “distal” refers to a position, direction, or portion of a device that is further away from the user and closer to the implantation site. Thus, for example, proximal motion of a device is motion of the device away from the implantation site and toward the user (e.g., out of the patient's body), while distal motion of the device is motion of the device away from the user and toward the implantation site (e.g., into the patient's body). The terms “longitudinal” and “axial” refer to an axis extending in the proximal and distal directions, unless otherwise expressly defined.

As used herein, “e.g.” means “for example,” and “i.e.” means “that is.”

### Overview of the Disclosed Technology

As introduced above, a leaflet assembly comprising a plurality of leaflets can be mounted on an inside of a frame of a prosthetic heart valve. The leaflet assembly is configured to regulate blood flow through the prosthetic heart valve, from an inflow end to an outflow end of the prosthetic heart valve. Each leaflet can comprise a main body with a cusp edge portion and a free or outflow edge (or edge portion). Each leaflet can also comprise a set of commissure tabs extending from the main body on opposite sides of the leaflet. The leaflets can be secured to one another at adjacent commissure tabs to form commissures that are then secured to the frame of the prosthetic heart valve. The cusp edge portion of each leaflet can also be coupled to struts of the frame (either directly or indirectly by an inner skirt), and in some examples the inflow end or end portion of the cusp edge portion can be secured to the inflow end of the frame. The main body of each leaflet, between the free (or outflow) edge of the leaflet and the cusp edge portion can be movable and opens and closes (or coapts) during operation of the prosthetic heart valve (during systole and diastole).

In some instances, prosthetic heart valves can experience unwanted tissue overgrowth at the inflow ends of the leaflets (e.g., the end disposed proximate to or at the inflow end of the frame, such as at inflow apices of the frame). In some examples, the leaflets can be secured to an inner skirt along their cusp edge portions, and the inner skirt (and the leaflets secured thereto) can be secured to the frame. In such instances, blood may accumulate in a pocket formed between the leaflets and the skirt. Further, in some instances, the free or outflow edges of the leaflets may not fully coapt together when assembled inside the frame and implanted in a patient.

Disclosed herein are leaflets that result in improved mobility of the leaflet and improved coaptation (e.g., closing) of the free or outflow edges of the leaflets when the prosthetic valve is assembled and implanted in a patient. The leaflets described herein may also result in reduced tissue overgrowth at the inflow ends of the leaflets, and/or improved washout of blood that may accumulate in any pockets formed between the leaflets and the skirt of the prosthetic heart valve. In some examples, the leaflets described herein can have a larger span (or width) and a more three-dimensional and concave shape, thereby resulting in increased mobility of the leaflet when the prosthetic heart valve is implanted in a patient. As a result, the efficiency of the prosthetic heart valve including the leaflets can be improved.

Prosthetic valves disclosed herein can be radially compressible and expandable between a radially compressed state and a radially expanded state. Thus, the prosthetic valves can be crimped on or retained by an implant delivery apparatus in the radially compressed state while being advanced through a patient's vasculature on the delivery apparatus. The prosthetic valve can be expanded to the radially expanded state once the prosthetic valve reaches the implantation site. It is understood that the prosthetic valves disclosed herein may be used with a variety of implant delivery apparatuses and can be implanted via various delivery procedures, examples of which will be discussed in more detail later.

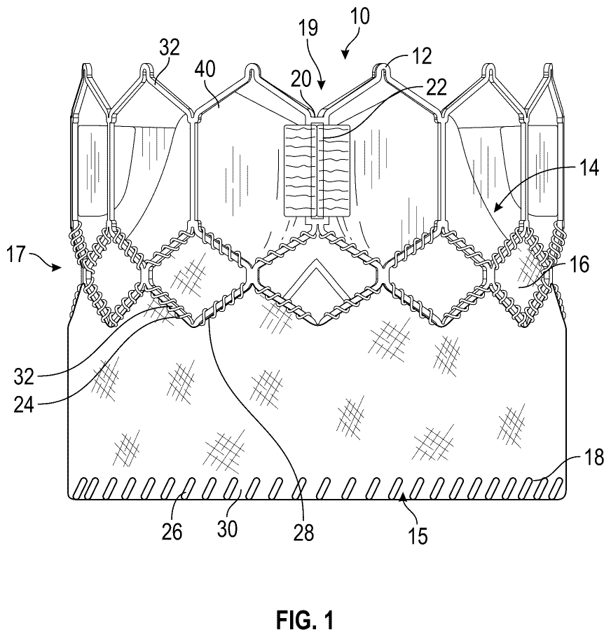

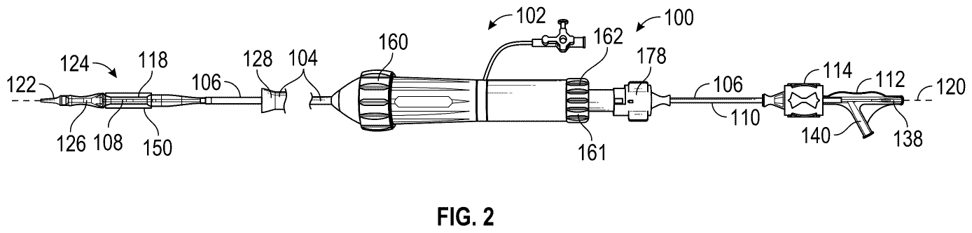

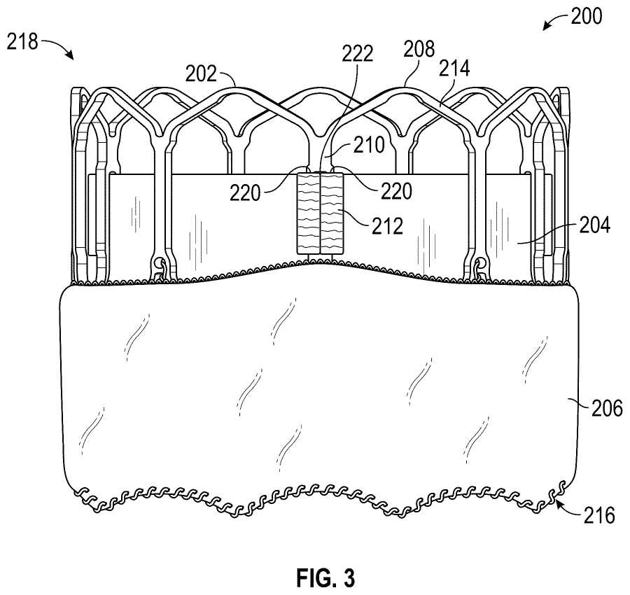

FIG. 1 illustrates an exemplary prosthetic device (for example, prosthetic heart valve) comprising a frame, leaflets secured on an inside of the frame, and an outer skirt disposed around an outer surface of the frame. In some examples, the frame can comprise commissure windows configured to receive adjacent sides of adjacent leaflets, thereby forming commissures that are secured to the frame. The prosthetic device can be advanced through a patient's vasculature, such as to a native heart valve, by a delivery apparatus, such as the exemplary delivery apparatus shown in FIG. 2. FIG. 3 shows an example of an exemplary prosthetic device (for example, prosthetic heart valve) that comprises a plurality of leaflets and can be delivered using the delivery apparatus of FIG. 2.

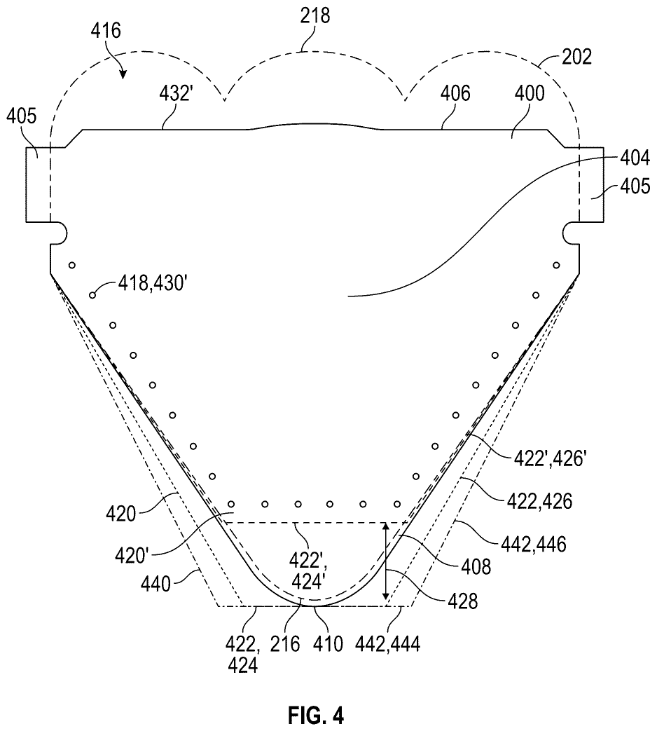

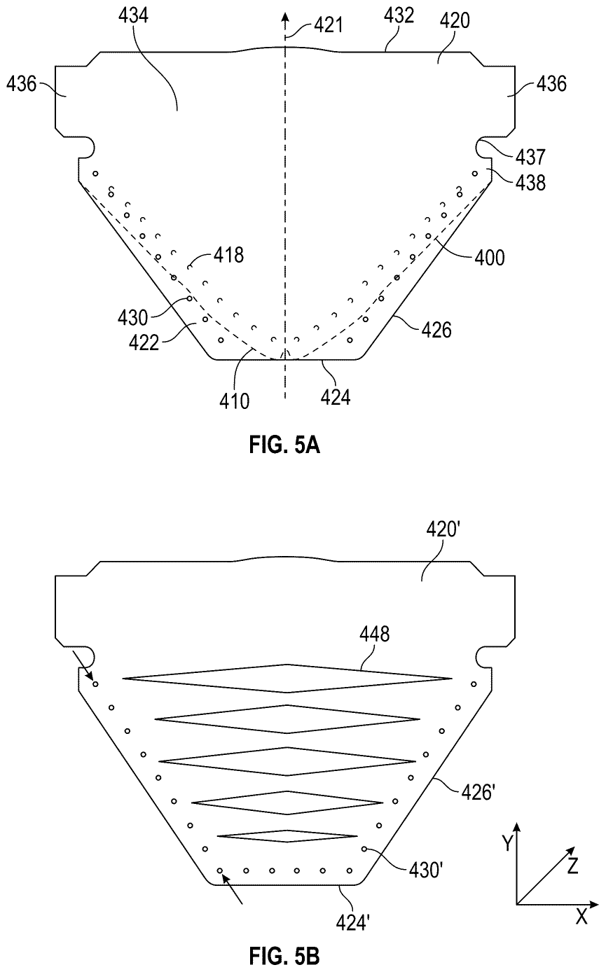

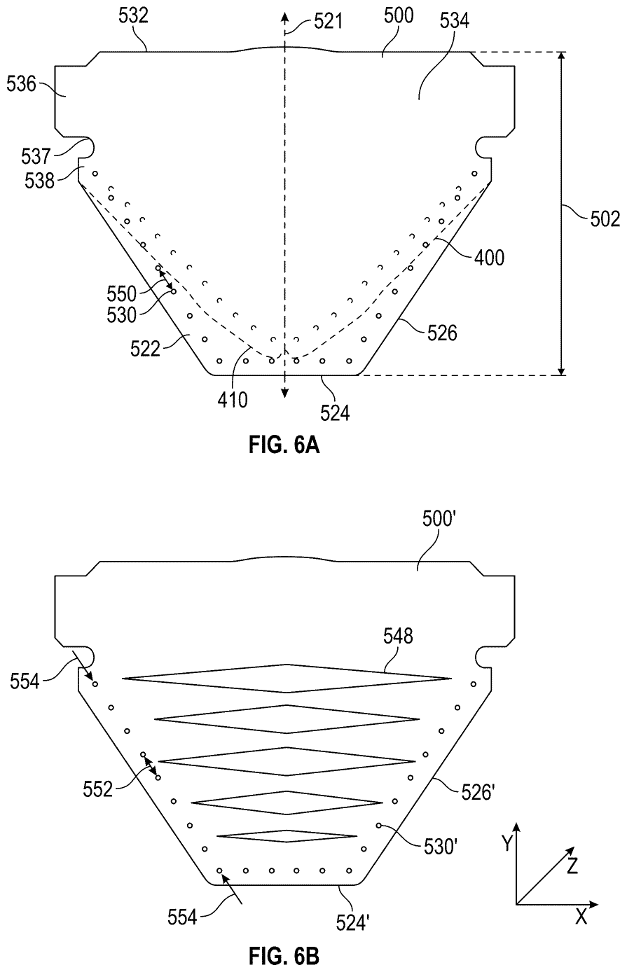

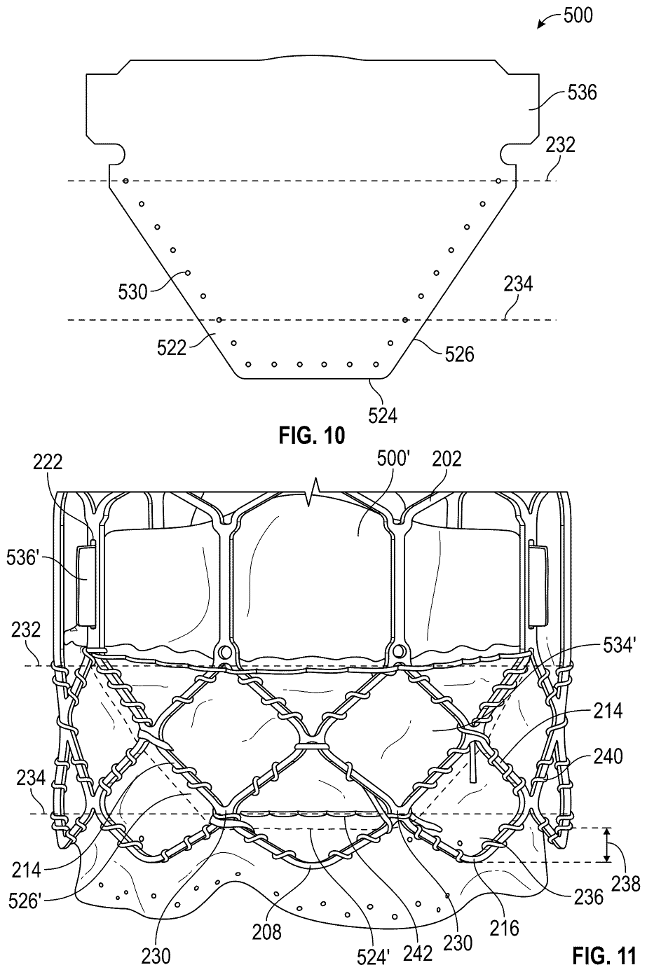

Exemplary leaflets for a prosthetic valve (such as the prosthetic valves shown in FIG. 1 or 3) that have a trapezoidal-shaped cusp edge are shown superimposed over a leaflet with a more traditional, curved cusp edge shape and a frame for the prosthetic leaflet in FIG. 4. In some examples, a leaflet can have a cusp edge with a more trapezoidal shape, thereby resulting in a straight (or laterally extending) inflow edge portion of the cusp edge that can be attached to a frame such that it is offset from the inflow end, and/or inflow apices, of the frame. The angled side edges of the cusp edge of the leaflet can be distanced farther away from one another than a traditional leaflet. Thus, when the cusp edge of the leaflet is coupled to the frame, along the struts following a shape of the leaflet scallop line (of the more traditional leaflet), the angled side edges and inflow edge portion of the cusp edge are moved towards one another, thereby compressing the leaflet material of the leaflet together and causing a main body of the leaflet to bow or bulge outwards. FIGS. 5A, 6A, 7A, 8A, and 9A show different designs for such trapezoidal-shaped leaflets in comparison to a more traditional leaflet with a curved cusp edge, and FIGS. 5B, 6B, 7B, 8B, and 9B show the compressed leaflet material for each respective leaflet design resulting from compressing and/or moving the edges of the cusp edge of the leaflet toward one another when coupling to the frame, as described above. FIG. 10 shows the new leaflet of FIG. 6A alone and FIG. 11 shows the leaflet of FIG. 10 attached to the frame, with the dashed lines in FIGS. 10 and 11 showing the location of the designated portions of the leaflet on the frame.

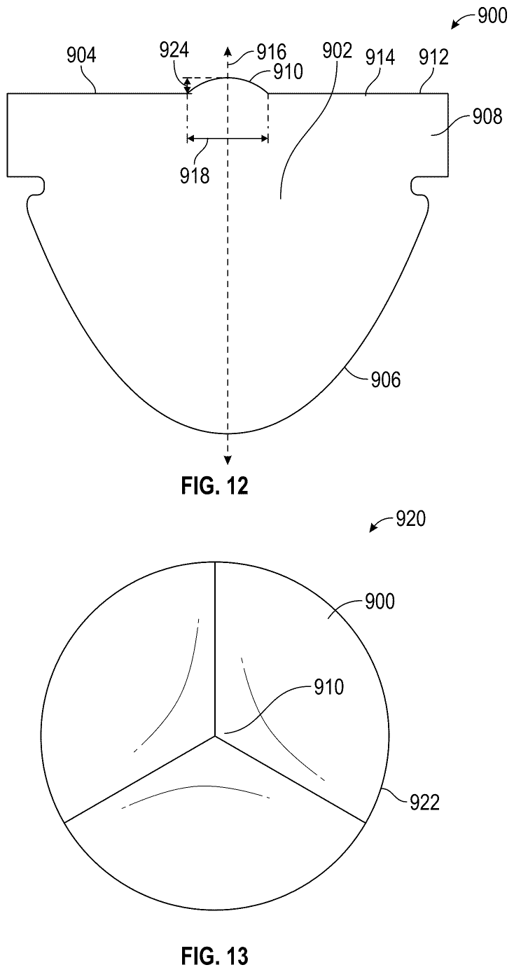

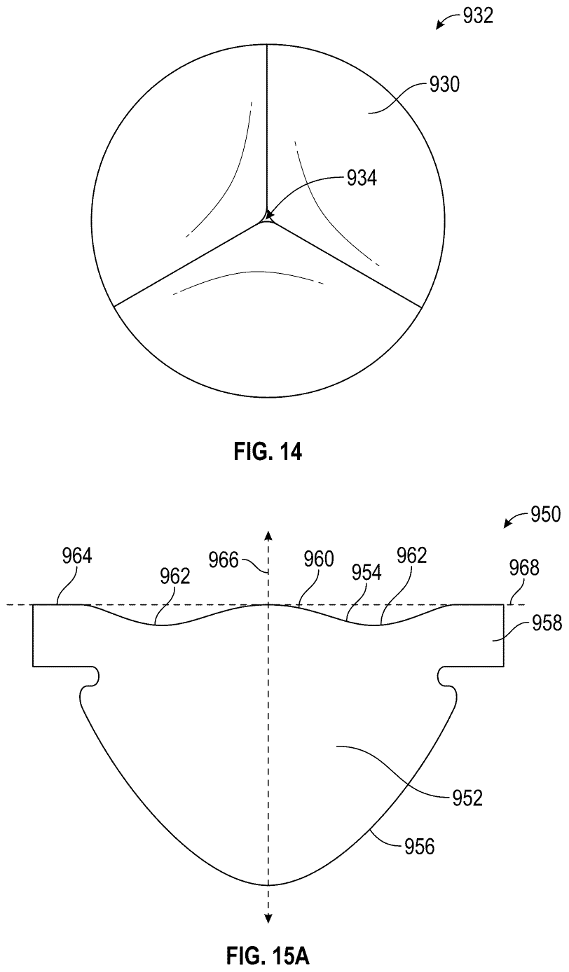

In some examples, a leaflet can comprise a cusp edge portion with a central bump or protrusion that is configured to allow leaflets mounted in a prosthetic valve to fully coapt together (with little to no gaps between their outflow edge portions) when implanted in a patient, as shown in FIG. 13 (as compared to a prosthetic valve having leaflets without a central bump, as shown in FIG. 14). FIGS. 12 and 15A-17 present exemplary leaflets comprising a cusp edge portion with a central bump or protrusion.

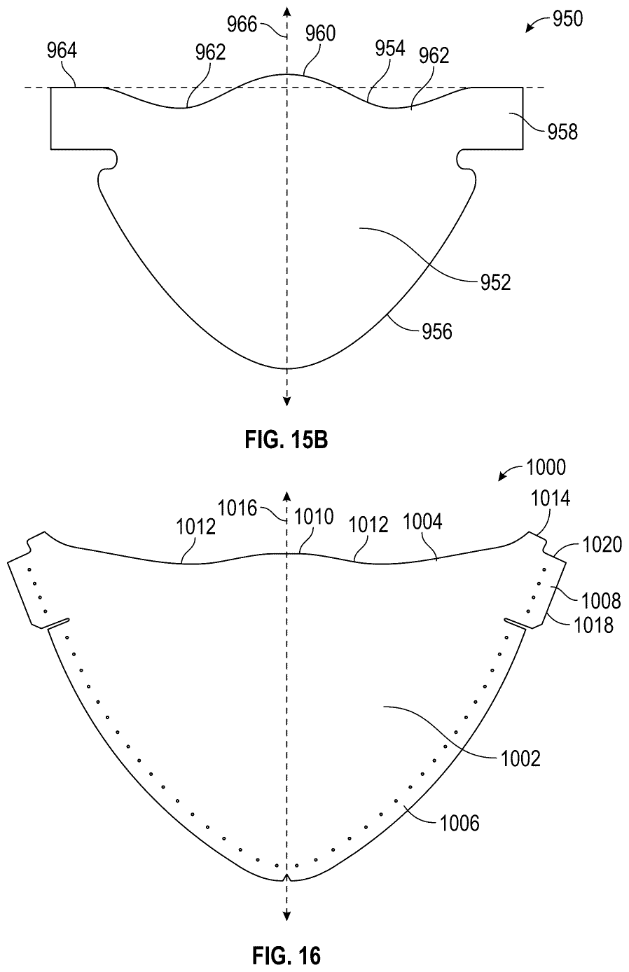

In some examples, the cusp edge portion of the leaflet can comprise depressed regions on both sides of the central bump, as depicted in the examples of FIGS. 15A-17.

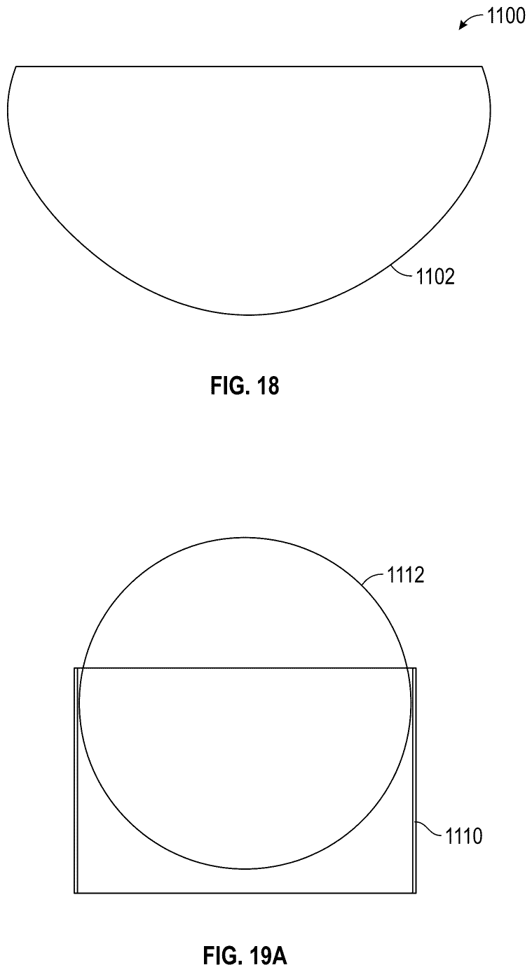

In some examples, a leaflet can have a rounded or circular cusp edge, as shown in FIG. 18. Traditionally, leaflets are cut in a flattened configuration (or 2D configuration since it is cut on a flat plane). However, when the leaflet is mounted within and coupled to an annular frame of a prosthetic valve, the rounded or circular cusp edge is no longer rounded circular, but instead distorted due to the 3D shape assumed by the leaflet after attachment.

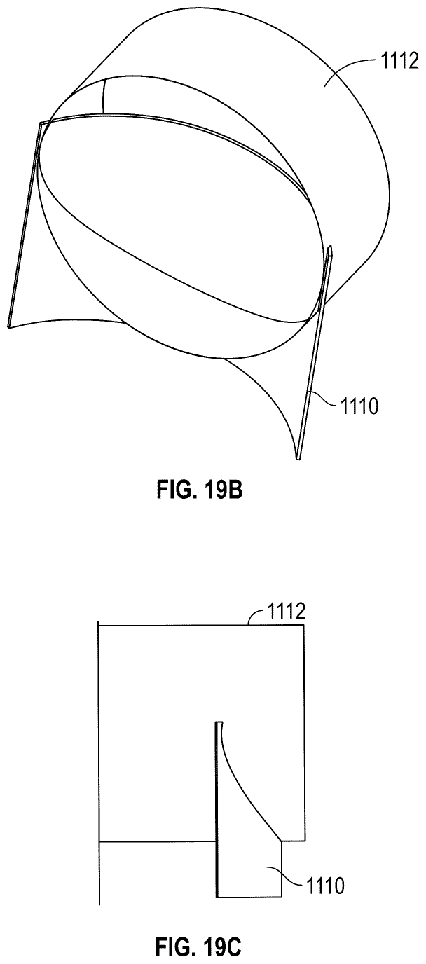

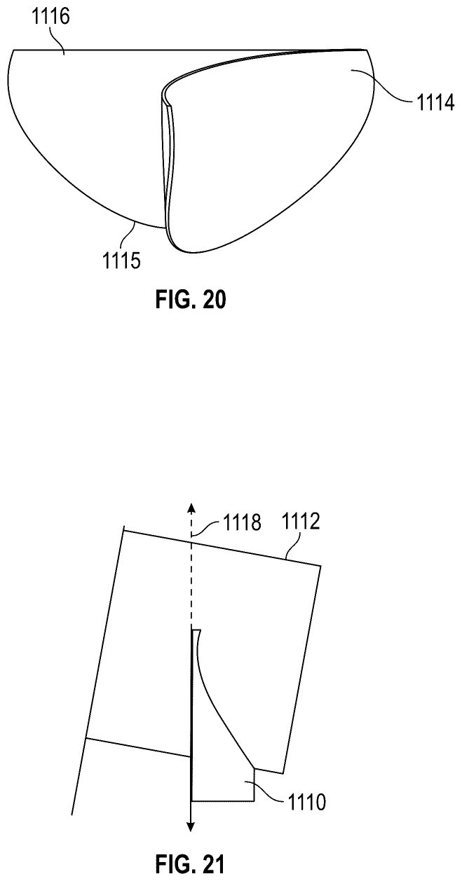

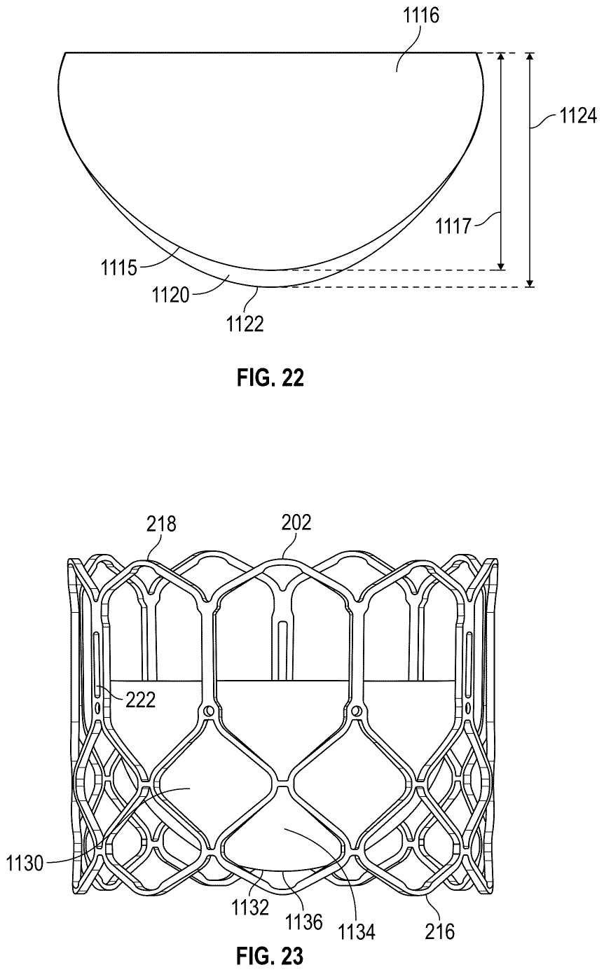

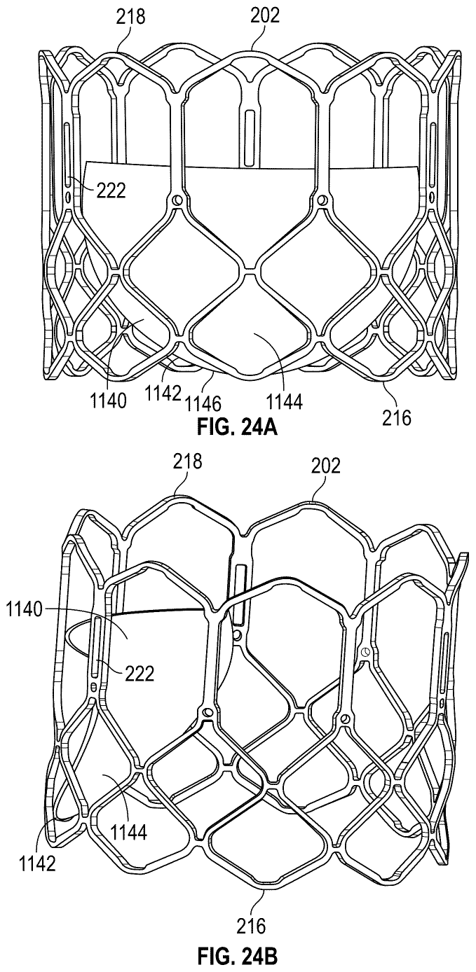

To create a 3D leaflet with a rounded or circular cusp edge when mounted within and to an annular frame of a prosthetic valve, a 3D leaflet can be created virtually (such as by using computer aided design software) with a cutting cylinder intersecting a leaflet cylinder (as shown in FIGS. 19A-19C and 21. The virtual 3D leaflet shape can then be flattened to form a 2D leaflet shape that is used to form a 2D leaflet cutting template for cutting a leaflet material in a flattened configuration (as shown in FIGS. 20 and 22). When the leaflets are coupled to an annular frame, as shown in FIGS. 23-24B, they then assume a 3D configuration with a rounded or circular cusp edge and a concave shape to at least a portion of the main body of the leaflet.

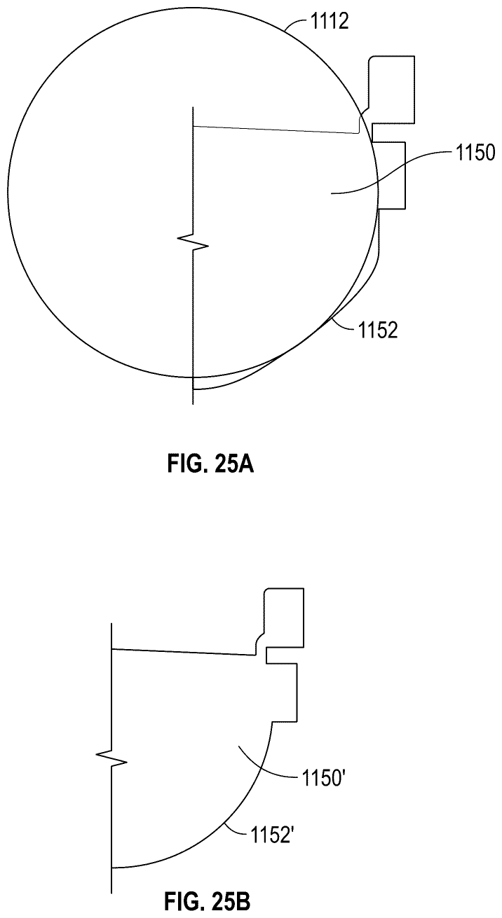

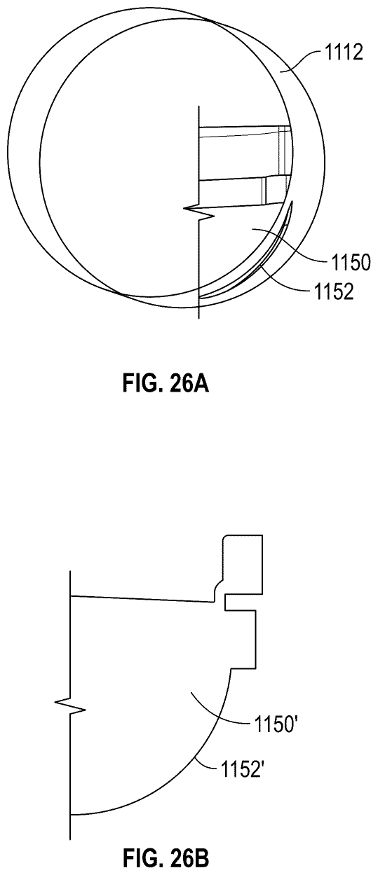

Examples of modifying a pre-existing (or previously cut) leaflet to have a rounded or circular cusp edge are shown in FIGS. 25A-26B.

### Examples of the Disclosed Technology

FIG. 1 shows an exemplary prosthetic valve 10, according to one example. Any of the prosthetic valves disclosed herein are adapted to be implanted in the native aortic annulus, although in some examples they can be adapted to be implanted in the other native annuluses of the heart (the pulmonary, mitral, and tricuspid valves). The disclosed prosthetic valves also can be implanted within vessels communicating with the heart, including a pulmonary artery (for replacing the function of a diseased pulmonary valve, or the superior vena cava or the inferior vena cava (for replacing the function of a diseased tricuspid valve) or various other veins, arteries and vessels of a patient. The disclosed prosthetic valves also can be implanted within a previously implanted prosthetic valve (which can be a prosthetic surgical valve or a prosthetic transcatheter heart valve) in a valve-in-valve procedure.

In some examples, the disclosed prosthetic valves can be implanted within a docking or anchoring device that is implanted within a native heart valve or a vessel. For example, in one example, the disclosed prosthetic valves can be implanted within a docking device implanted within the pulmonary artery for replacing the function of a diseased pulmonary valve, such as disclosed in U.S. Publication No. 2017/0231756, which is incorporated by reference herein. In some examples, the disclosed prosthetic valves can be implanted within a docking device implanted within or at the native mitral valve, such as disclosed in PCT Publication No. WO2020/247907, which is incorporated herein by reference. In some examples, the disclosed prosthetic valves can be implanted within a docking device implanted within the superior or inferior vena cava for replacing the function of a diseased tricuspid valve, such as disclosed in U.S. Publication No. 2019/0000615, which is incorporated herein by reference.

The prosthetic valve 10 can have four main components: a stent or frame 12, a valvular structure 14, an inner skirt 16, and a perivalvular outer sealing member or outer skirt 18. The prosthetic valve 10 can have an inflow end portion 15, an intermediate portion 17, and an outflow end portion 19.

The valvular structure 14 can comprise three leaflets 40, collectively forming a leaflet structure, which can be arranged to collapse in a tricuspid arrangement, although in some examples there can be greater or fewer number of leaflets (for example, one or more leaflets 40). The leaflets 40 can be secured to one another at their adjacent sides to form commissures 22 of the valvular (or leaflet) structure 14. The lower edge of valvular structure 14 can have an undulating, curved scalloped shape and can be secured to the inner skirt 16 by sutures (not shown). In some examples, the leaflets 40 can be formed of pericardial tissue (for example, bovine pericardial tissue), biocompatible synthetic materials, or various other suitable natural or synthetic materials as known in the art and described in U.S. Pat. No. 6,730,118, which is incorporated by reference herein.

The frame 12 can be formed with a plurality of circumferentially spaced slots, or commissure windows 20 that are adapted to mount the commissures 22 of the valvular structure 14 to the frame. The frame 12 can be made of any of various suitable plastically-expandable materials (for example, stainless steel, etc.) or self-expanding materials (for example, Nitinol), as known in the art. When constructed of a plastically-expandable material, the frame 12 (and thus the prosthetic valve 10) can be crimped to a radially collapsed (or compressed) configuration on a delivery catheter and then expanded inside a patient by an inflatable balloon or equivalent expansion mechanism to a radially expanded configuration. When constructed of a self-expandable material, the frame 12 (and thus the prosthetic valve 10) can be crimped to a radially collapsed configuration and restrained in the collapsed configuration by insertion into a sheath or equivalent mechanism of a delivery catheter. Once inside the body, the prosthetic valve can be advanced from the delivery sheath, which allows the prosthetic valve to expand to its functional size.

Suitable plastically-expandable materials that can be used to form the frame 12 include, metal alloys, polymers, or combinations thereof. Example metal alloys can comprise one or more of the following: nickel, cobalt, chromium, molybdenum, titanium, or other biocompatible metal. In some examples, the frame 12 can comprise stainless steel. In some examples, the frame 12 can comprise cobalt-chromium. In some examples, the frame 12 can comprise nickel-cobalt-chromium. In some examples, the frame 12 comprises a nickel-cobalt-chromium-molybdenum alloy, such as MP35N™ (tradename of SPS Technologies), which is equivalent to UNS R30035 (covered by ASTM F562-02). MP35N™/UNS R30035 comprises 35% nickel, 35% cobalt, 20% chromium, and 10% molybdenum, by weight.

The frame 12 can comprise a plurality of interconnected struts 32 that form cells (or openings or void spaces) in the frame.

In some examples, as shown in FIG. 1 an upper edge portion 28 (also referred to as an outflow edge portion) of the outer skirt 18 can be secured to the frame 12 by stitches 24 and a lower edge portion 30 (also referred to as an inflow edge portion) of the outer skirt 18 can be secured to the frame 12 by stitches 26 extending along the inflow end portion 15 of the prosthetic valve 10. For example, the stitches 24 can wrap around struts 32 of the frame 12 forming a circumferentially extending row of struts 32 at the intermediate portion 17 of the prosthetic valve 10. Further, in some examples, the stitches 26 can wrap around struts 32 of the frame 12 forming a circumferentially extending row of struts 32 at the inflow end portion 15 of the prosthetic valve 10. In some instances, the upper edge portion 28 can be secured to struts 32 that are closer to the outflow end portion 19 of the frame 12 (such as the circumferentially extending row of struts 32 forming inflow ends of the row of cells disposed at the outflow end portion 19.

FIG. 2 shows a delivery apparatus 100, according to an example, that can be used to implant an expandable prosthetic heart valve (for example, the prosthetic heart valve 10 of FIG. 1 and/or any of the other prosthetic heart valves described herein). In some examples, the delivery apparatus 100 is specifically usable or adapted for use in introducing a prosthetic valve into a heart.

The delivery apparatus 100 in the illustrated example of FIG. 2 is a balloon catheter comprising a handle 102 and a steerable, outer shaft 104 extending distally from the handle 102. The delivery apparatus 100 can further comprise an intermediate shaft 106 (which also may be referred to as a balloon shaft) that extends proximally from the handle 102 and distally from the handle 102, the portion extending distally from the handle 102 also extending coaxially through the outer shaft 104. Additionally, the delivery apparatus 100 can further comprise an inner shaft 108 extending distally from the handle 102 coaxially through the intermediate shaft 106 and the outer shaft 104 and proximally from the handle 102 coaxially through the intermediate shaft 106.

The outer shaft 104 and the intermediate shaft 106 can be configured to translate (move) longitudinally, along a central longitudinal axis 120 of the delivery apparatus 100, relative to one another to facilitate delivery and positioning of a prosthetic valve at an implantation site in a patient's body.

The intermediate shaft 106 can include a proximal end portion 110 that extends proximally from a proximal end of the handle 102, to an adaptor 112. A rotatable knob 114 can be mounted on the proximal end portion 110 and can be configured to rotate the intermediate shaft 106 around the central longitudinal axis 120 and relative to the outer shaft 104.

The adaptor 112 can include a first port 138 configured to receive a guidewire therethrough and a second port 140 configured to receive fluid (for example, inflation fluid) from a fluid source. The second port 140 can be fluidly coupled to an inner lumen of the intermediate shaft 106.

The intermediate shaft 106 can further include a distal end portion that extends distally beyond a distal end of the outer shaft 104 when a distal end of the outer shaft 104 is positioned away from an inflatable balloon 118 of the delivery apparatus 100. A distal end portion of the inner shaft 108 can extend distally beyond the distal end portion of the intermediate shaft 106.

The balloon 118 can be coupled to the distal end portion of the intermediate shaft 106.

In some examples, a distal end of the balloon 118 can be coupled to a distal end of the delivery apparatus 100, such as to a nose cone 122 (as shown in FIG. 2), or to an alternate component at the distal end of the delivery apparatus 100 (for example, a distal shoulder). An intermediate portion of the balloon 118 can overlay a valve mounting portion 124 of a distal end portion of the delivery apparatus 100 and a distal end portion of the balloon 118 can overly a distal shoulder 126 of the delivery apparatus 100. The valve mounting portion 124 and the intermediate portion of the balloon 118 can be configured to receive a prosthetic heart valve in a radially compressed state. For example, as shown schematically in FIG. 2, a prosthetic heart valve 150 (which can be one of the prosthetic valves described herein) can be mounted around the balloon 118, at the valve mounting portion 124 of the delivery apparatus 100.

The balloon shoulder assembly, including the distal shoulder 126, is configured to maintain the prosthetic heart valve 150 (or other medical device) at a fixed position on the balloon 118 during delivery through the patient's vasculature.

The outer shaft 104 can include a distal tip portion 128 mounted on its distal end. The outer shaft 104 and the intermediate shaft 106 can be translated axially relative to one another to position the distal tip portion 128 adjacent to a proximal end of the valve mounting portion 124, when the prosthetic valve 150 is mounted in the radially compressed state on the valve mounting portion 124 (as shown in FIG. 2) and during delivery of the prosthetic valve to the target implantation site. As such, the distal tip portion 128 can be configured to resist movement of the prosthetic valve 150 relative to the balloon 118 proximally, in the axial direction, relative to the balloon 118, when the distal tip portion 128 is arranged adjacent to a proximal side of the valve mounting portion 124.

An annular space can be defined between an outer surface of the inner shaft 108 and an inner surface of the intermediate shaft 106 and can be configured to receive fluid from a fluid source via the second port 140 of the adaptor 112. The annular space can be fluidly coupled to a fluid passageway formed between the outer surface of the distal end portion of the inner shaft 108 and an inner surface of the balloon 118. As such, fluid from the fluid source can flow to the fluid passageway from the annular space to inflate the balloon 118 and radially expand and deploy the prosthetic valve 150.

An inner lumen of the inner shaft can be configured to receive a guidewire therethrough, for navigating the distal end portion of the delivery apparatus 100 to the target implantation site.

The handle 102 can include a steering mechanism configured to adjust the curvature of the distal end portion of the delivery apparatus 100. In the illustrated example, for example, the handle 102 includes an adjustment member, such as the illustrated rotatable knob 160, which in turn is operatively coupled to the proximal end portion of a pull wire. The pull wire can extend distally from the handle 102 through the outer shaft 104 and has a distal end portion affixed to the outer shaft 104 at or near the distal end of the outer shaft 104. Rotating the knob 160 can increase or decrease the tension in the pull wire, thereby adjusting the curvature of the distal end portion of the delivery apparatus 100. Further details on steering or flex mechanisms for the delivery apparatus can be found in U.S. Pat. No. 9,339,384, which is incorporated by reference herein.

The handle 102 can further include an adjustment mechanism 161 including an adjustment member, such as the illustrated rotatable knob 162, and an associated locking mechanism including another adjustment member, configured as a rotatable knob 178. The adjustment mechanism 161 is configured to adjust the axial position of the intermediate shaft 106 relative to the outer shaft 104 (for example, for fine positioning at the implantation site). Further details on the delivery apparatus 100 can be found in PCT Application No. PCT/US2021/047056, which is incorporated by reference herein.

FIG. 3 shows a prosthetic valve 200 comprising a radially expandable and/or compressible annular frame 202, a plurality of leaflets 204 mounted within the frame 202, and an outer skirt 206 secured to and around an outer surface of the frame 202. The frame 202 can comprise a plurality of interconnected angled struts 214. The angled struts 214 are arranged in a plurality of circumferentially extending rows of angled struts 214, with the rows being arrayed along the length of the frame 202 between an inflow end 216 and an outflow end 218 of the frame 202. A first circumferentially extending row of angled struts 214 at the outflow end 218 of the frame 202 is visible in FIG. 3, and additional circumferentially extending rows of angled struts 214 of the frame 202 can be seen in FIG. 11 and FIGS. 28-29B, which are described further below.

The frame 202 can comprise a plurality of apices 208 (or apex regions) that are spaced circumferentially apart around the inflow end 216 and the outflow end 218 of the frame 202 (only the apices 208 at the outflow end 218 are visible in FIG. 3; however, the apices 208 at the inflow end 216 are shown in FIG. 11, which is described further below). Each apex 208 is formed at a junction between two angled struts 214 at either the inflow end 216 or the outflow end 218.

The frame 202 comprises a plurality of axially extending posts 210, some of which define commissure windows therein. For example, the axially extending posts 210 that define commissure windows therein can comprise axially extending window struts 220 (which can also be referred to herein as commissure supports or commissure support posts) that define a commissure window 222 therebetween. Commissure tabs of adjacent leaflets 204 can be paired together and extend through the commissure windows 222, thereby forming commissures 212 secured to the frame 202 which can protrude radially outward from the frame 202. In some examples, the prosthetic valve 200 can be a balloon expandable valve and can be used with the delivery apparatus 100 of FIG. 2. Additional details on the prosthetic valve 200 can be found in PCT Publication No. WO/2022/226147, which is incorporated by reference herein.

FIG. 4 is a schematic of a first leaflet 400 (shown with a solid line) coupled to and/or mounted within a portion of the frame 202 (which is shown schematically with a dashed line). However, the frame 202 can be replaced by any of the frames described herein, such as the frame 12 of FIG. 1, or similar frames for prosthetic heart valves.

As shown in FIG. 4, the first leaflet 400 comprises a main body 404 with an outflow edge portion 406 (or free edge portion) and a cusp edge portion 408. The cusp edge portion 408 can have a more conventional shape which includes a curved lower or inflow end 410 that is coupled to the frame 202 along the inflow end 216 of the frame (adjacent to inflow apices 208 of the frame 202, for example).

The first leaflet 400 can further comprise opposing commissure tabs 405 disposed on opposite sides of the main body 404.

The outflow edge portion 406 (or outflow edge or free edge) of the first leaflet 400 extends along a portion of the frame 202 which is distanced from the outflow end 218 of the frame 202, leaving exposed (or uncovered) portions of the upper cells 416 of the frame 202 through which blood perfusion and coronary access may be maintained.

In some examples, coupling of the first leaflet 400 to the frame 202 is performed by passing a suture (or other fastener) along the scallop line of the first leaflet 400 and corresponding struts of the frame 202 and through the cusp edge portion 408 of the first leaflet 400. For example, the suture can pass in-and-out through apertures 418 (or penetration points) in the cusp edge portion 408, as shown in FIG. 4, and around struts of the frame 202 extending along the cusp edge portion 408.

In some examples, coupling of the first leaflet 400 to the frame 202 is performed by passing a suture (or other fastener) along the scallop line, or cusp edge portion 408, of the first leaflet 400, through apertures 418 and an inner skirt disposed against the first leaflet 400 in an in-and-out pattern to form a plurality of connecting stitches securing the cusp edge portion 408 to the inner skirt (such as the inner skirt 236 shown in FIG. 11). A plurality of stitches can loop around the plurality of connecting stitches and around struts of the frame 202 extending along the scallop line, thereby coupling the first leaflet 400 to the frame 202 through the inner skirt.

As used herein, the term “scallop line” can refer to a shape of the cusp edges of the leaflets of a prosthetic heart valve or a path that tracks the curvature of the cusp edges of the leaflets (since it undulates or looks like a scallop in some cases). In some examples, the cusp edges of the leaflets are attached or fixed relative to the frame with stitches that follow the scallop line (directly to struts of the frame or indirectly by stitching the cusp edges to an inner skirt). In some examples, as described below, a leaflet can have a trapezoidal cusp edge having straight sides and therefore the leaflet assembly does not have a true, curved scallop shape along the cusp edges of the leaflets. Nonetheless, for convenience, the path along the cusp edges of such leaflets or the overall shape of a leaflet assembly along the cusp edges can be referred to as a scallop line. The as-cut and on-frame scallop line of the leaflets can, in some examples, be different, such as for the second leaflet 420, as described further below. Further, a “scallop line of the frame” or “an attachment line” can refer to an undulating line along the struts of the frame to which the cusp edges of the leaflets are attached, such as by stitching. “An attachment line” can also refer to a path along which the cusp edges of the leaflets are stitched or otherwise attached to an inner skirt, which in turn is attached to the frame.

To reduce a likelihood of tissue overgrowth along the inflow end of the leaflet (e.g., the inflow end 410 of first leaflet 400), the inflow end of the leaflet can be offset upwards (or downstream), away from the inflow end 216 of the frame 202. However, the outflow edge portion 406 can be maintained at the same level or location as shown in FIG. 4, thereby maintaining the same open or exposed area of the upper cells 416 downstream of the first leaflet 400.

In some examples, and as used herein, the inflow end 216 of the frame 202 can be defined or formed by the inflow apices 208. Thus, the inflow end of the leaflet being offset away from the inflow end 216 of the frame 202 can refer to the inflow end of the leaflet being offset away from the inflow apices 208 of the frame 202. In this way, when one or more skirts are disposed around the frame, as shown in FIGS. 1 and 11, for example, the inflow ends of the leaflets can be spaced away from an inflow end of the prosthetic valve.

To achieve this leaflet arrangement on the frame, a second leaflet 420 can be shaped to have a more trapezoidal shape along its cusp edge portion 422, as depicted with a dashed line in FIG. 4. For example, the cusp edge portion 422 of the second leaflet 420 comprises a relatively linear or straight inflow edge 424 and two angled side edges 426 which form a shape that can be similar to a portion of an isosceles trapezoid. As shown in FIG. 4, the inflow edge 424 distances the side edges 426 farther away from each other relative to the side edges of the cusp edge portion 408 of the first leaflet 400.

When attaching or coupling the second leaflet 420 to the frame 202, the inflow edge 424 and the side edges 426 of the second leaflet 420 are translated and compressed toward the original scallop line of the first leaflet 400, which aligns with the struts of the frame 202, and such that the inflow edge 424 is translated downstream (upwards in the view of FIG. 4) and farther away from the inflow end 216 of the frame 202. Stated differently, the side edges 426 are pushed toward each other and the inflow edge 424 is pushed toward the outflow edge portion 406 to align the side edges 426 and the inflow edge 424 with the intended attachment line along the frame, which causes the leaflet to undergo compression buckling and deflect out of plane from its pre-attached, off-frame configuration. The compressed or on-frame (e.g., when coupled to the struts of the frame 202) configuration of the second leaflet 420 (as opposed to the as-cut and off-frame configuration) is shown by another dashed line in FIG. 4 and represented by prime symbols of the same number (e.g., second leaflet 420′ is the second leaflet 420 in the compressed and on-frame configuration).

As introduced above, an outflow edge portion 432′ of the second leaflet 420′ can be maintained at the same level or location on the frame 202 as the first leaflet 400, thereby maintaining the same open or exposed area of the upper cells 416 downstream of the leaflet 420′.

In some examples, a length of the inflow edge 424 can be determined by the lowermost non-apical junctions 230 adjacent to the inflow end 216 of the frame, on both sides of an inflow apex 208 (or apex region) of the frame 202 (as shown in FIG. 11, which is described in greater detail below). This enables the inflow edge 424 to be compressed along its length and offset away from the inflow end 216 and towards the outflow end 218 of the frame 202 by an axial distance 428 (as shown in FIG. 4).

In some examples, the distance 428 can be in a range of 1 to 3 mm, 1.5 to 2.5 mm, or about 2 mm.

As introduced above, since the as-cut or off-frame configuration of the second leaflet 420 results in the side edges 426 being distanced farther from each other, as compared to the first leaflet 400, attachment along the scallop line of the frame 202 can be performed by moving the side edges 426 of the second leaflet 420 closer to each other, such that apertures 430′ (or suture penetration points) along the scallop line remain in the same position as those of the apertures 418 of the first leaflet 400.

The axial distance 428 which the inflow edge 422′ is offset from the inflow end 216 of the frame 202 when attached to the frame 202 results in an amount of compression of the leaflet material in a range of about 5-25%. This compression can be referred to as longitudinal compression, or compression in an axial direction that is defined between the outflow edge portion 432′ and inflow edge 424′ of the leaflet 420.

In some examples, due to a compressibility of the leaflet material (such as pericardial tissue), moving the side edges 426 of the leaflet 420 toward one another to couple the leaflet 420 to the frame 202 can result in a smaller effect on the movable portion of the leaflet 420 (e.g., the body). For example, as explained below, the extent to which the body bulges or pops outward (e.g., in a z direction, as shown in FIG. 5B) can be smaller than in leaflets where the side edges 426 are spaced farther apart in the as-cut configuration.

Thus, in some examples, it may be beneficial to add leaflet material between the side edges 426 to enhance the leaflet's motion as it moves between the open and closed states (when a prosthetic valve including the leaflets is implanted and operating in vivo). FIG. 4 shows an exemplary third leaflet 440 (shown with another dashed line) that is similar to the leaflet 420 but distances the side edges 446 of the cusp edge portion 442 farther apart from each other. Thus, the inflow edge 444 of the third leaflet 440 is longer than the inflow edge 424 of the second leaflet 420, as shown in FIG. 4.

In some examples, the inflow edge 444 of the third leaflet 440 is about 0.25-1.5 mm, or about 0.5-1 mm longer than the inflow edge 424 of the second leaflet 420.

Attachment of the third leaflet 440 to the frame 202 (along the scallop line) can be performed in a similar manner, moving or pressing the side edges 446 closer to each other to allow attachment along similarly positioned apertures or suture penetration points.

However, the longer length of the inflow edge 444, and thus the longer distance between the side edges 446 will result in additional compressed lateral material of the third leaflet 440, which in turn can advantageously improve leaflet mobility to facilitate blood washout from a “pocket” that may be formed between the leaflet and the skirt, as described above.

FIGS. 5A, 6A, 7A, 8A, and 9A show various shapes for cusp edge portions of leaflets, where the leaflets are shown in their as-cut and off-frame configuration. The leaflets are shown superimposed over the first leaflet 400 described above for comparison, wherein the leaflet 400 has a more conventional, curved inflow edge of the cusp edge portion. FIGS. 5B, 6B, 7B, 8B, and 9B show the various leaflets in their compressed or on-frame configurations, and schematically depict the compressed leaflet material (by the diamond or trapezoidal shapes in the main body of the leaflet) for each leaflet shape that results from compressing the leaflet and/or moving the edges of the cusp edge portions toward one another to follow the scallop line of the frame (e.g., frame 202).

FIGS. 5A and 5B show the leaflet 420 described above with reference to FIG. 4. FIG. 5A shows the leaflet 420 in its as-cut or off-frame configuration, superimposed over the first leaflet 400 with the more conventional curved inflow end 410 of its cusp edge portion 408 (the first leaflet 400 is depicted with dashed lines for illustration purposes).

The leaflet 420 comprises a main body 434 with the outflow edge portion 432 (or free edge portion) and cusp edge portion 422. As described above, the cusp edge portion 422 includes the inflow edge 424 and side edges 426 which angle from opposite sides of the inflow edge 424.

The side edges 426 can extend from opposite sides of the inflow edge 424 towards respective commissure tabs 436.

In some examples, the side edges 426 can extend to smaller tabs 438 that are axially offset, relative to a central axis 421 of the leaflet 420, from the commissure tabs 436 by indented regions 437.

As discussed above and shown in FIG. 5A, the inflow edge 424 is straight (or relatively straight, which may include a slight wave or non-straight portion but still have an overall straight or linear shape) and distances the side edges 426 farther away from one another than the cusp edge portion 408 of the first leaflet 400. For example, as shown in FIG. 5A the side edges 426 (shown with solid lines) are disposed outside of the side edges of the cusp edge portion 408 of the first leaflet 400 (shown with dashed lines).

In some examples, the inflow edge 424 can be parallel to one or more straight portions of the outflow edge portion 632.

As a result of the spacing apart of the side edges 426, the leaflet 420 has more leaflet material, as compared to the first leaflet 400 (e.g., more leaflet material in its main body 434). Thus, when the leaflet 420 is coupled to the frame (e.g., frame 202) and moved into its compressed or on-frame configuration of the leaflet 420′, as shown in FIG. 5B, the leaflet 420 is compressed in the x direction (which can also be referred to as the lateral direction) and y direction (which can also be referred to as the axial or longitudinal direction), thereby resulting in compressed leaflet material 448, which is depicted schematically by the diamond regions in FIG. 5B. The compressed leaflet material 448 can result in changes in the z-direction of the leaflet 420′, such as causing the leaflet material in these regions to bulge, buckle, or bow outward (in the z direction), thereby increasing the concavity/convexity of the main body of the leaflet 420′. In this way, the leaflet 420′ can have a more three-dimensional (3D) shape, as compared to the leaflet 420 in its off-frame configuration and as compared to the first leaflet 400 in its on-frame configuration.

In some examples, the resulting compression of the leaflet 420′ is in a range of 15-20%, or about 20% at all but the two lowest apertures 430′ (the apertures 430′ along the inflow edge 424′).

As used herein, “compressed leaflet material” refers to regions in the material of the leaflet that are compressed or loaded along an axis or plane generally parallel to the inner and outer main surfaces of the leaflet (perpendicular to its thickness), such as along the X-axis or Y-axis of the leaflet. This can be accomplished by moving the side edges 426 toward one another to attach to the attachment line defined by the struts of the frame, and/or by offsetting the inflow edge 424 away from the inflow end of the frame (which moves the inflow edge 424 inward toward the outflow edge portion 432 of the leaflet 420). This may result in bulging, buckling, or popping out of the leaflet material in the main body 434 (similar to compression buckling of a panel), which can cause the main body 434 to have a concave/convex shape in the regions of the compressed and/or squeezed together leaflet material. This is depicted schematically in the noted figures by diamond or trapezoid shapes.

FIGS. 6A and 6B show a leaflet 500 in its as-cut or off-frame configuration and superimposed over the first leaflet 400 (shown in FIG. 6A) and in its compressed or on-frame configuration (represented by leaflet 500′, as shown in FIG. 6B).

The leaflet 500 may be similar to the leaflet 420 except it has a longer axial length 502, which is defined in a direction of a central longitudinal axis 521 of the leaflet 500. As a result, the inflow edge 524 of its cusp edge portion 522 is offset from the inflow end 410 of the first leaflet 400.

The same or similar to the leaflet 420, the leaflet 500 comprises a main body 534 with an outflow edge portion 532 (which may be the same or similar to the outflow edge portion 432) and the cusp edge portion 522. The cusp edge portion 522 includes the inflow edge 524 and side edges 526 which angle from opposite sides of the inflow edge 524 (the same or similar to side edges 426). Apertures 530 can be spaced apart along each of the side edges 526 and/or the inflow edge 524.

The leaflet 500 can comprise opposing commissure tabs 536 (which can be similar to the commissure tabs 436).

In some examples, the leaflet 500 includes smaller tabs 538 that are axially offset, relative to the central longitudinal axis 521, from the commissure tabs 536 by indented regions 537.

The inflow edge 524 of the leaflet 500 can be straight (or relatively straight, which may include a slight wave or non-straight portion but still have an overall straight or linear shape) and distances the side edges 526 farther away from one another than the cusp edge portion 408 of the first leaflet 400.

In some examples, a length of the inflow edge 524 can be varied (e.g., increased) to vary the spacing between the side edges 526, based on a desired amount of excess leaflet material for creating bulging or billowing (and a more concave and/or 3D shape) when attaching the leaflet 500 to the annular frame.

Similar to as described above for the leaflet 420, when the leaflet 500 is coupled to the frame (e.g., frame 202), the side edges 526 can be moved and/or compressed inward toward one another (in the x direction) to couple to struts 214 of the frame 202 along the attachment line of the frame, and the inflow edge 524 can be moved and/or compressed toward its outflow edge portion 532 to offset the inflow edge 524 from the inflow end 216 of the frame 202 (as depicted in FIG. 11, which is described further below). As a result, the leaflet 500′ is moved into its compressed and/or on-frame configuration, as shown in FIG. 6B. FIG. 6B shows the compressed leaflet material 548, which results in bulging and/or increased concavity/convexity of the main body 534 of the leaflet 500′, as described above. In this way, the leaflet 500′ can have a more 3D shape, as compared to the leaflet 500 in its off-frame configuration and as compared to the first leaflet 400 in its on-frame configuration.

In some examples, the resulting compression of the leaflet 500′ is in a range of 20-25%, or about 25% at all of the apertures 530. For example, because the leaflet 500 is longer than the leaflet 420, offsetting the inflow edge 524 from the inflow end of the frame by the same amount (e.g., approximately 2 mm) can result in increased compression and/or bulging of the material of the leaflet 500′ (as compared to the leaflet 420′).

In some examples, the apertures 530 can be pre-formed in the cusp edge portion 522 of the leaflet 500. In the as-cut leaflet 500 of FIG. 6A, the apertures 530 can be separated from one another by a first distance 550. The apertures 530 may be moved toward one another as the leaflet 500 is compressed and attached to the frame. As a result, when the leaflet 500′ is in its compressed or on-frame configuration (as shown in FIG. 6B), the apertures 530′ can be separated from one another by a second distance 552 that is smaller than the first distance 550. This compression and movement of the apertures 530′ toward one another is depicted schematically in FIG. 6B by the arrows. In this way, compression of the side edges 526′ along their length can occur when attaching to the frame, and thereby resulting is the apertures 530′ moving closer together along the side edges 526′.

In some examples, the first distance 550 is about 1.8 mm and the second distance 552 is about 1.2 mm.

Similar decreases in distance between the pre-formed apertures 130′ along the cusp edge portion can occur in the other leaflets described herein during compression and/or attachment of the cusp edge portion to the annular frame of the prosthetic valve (such as the leaflets 420, 700, and 880, as depicted schematically in FIGS. 5B, 8B, and 9B, respectively, by the arrows extending along the respective edges of the cusp edge portion).

In some examples, when attaching to the frame, the inflow edge 524′ can also be compressed along its length (in the x direction or circumferential direction of the frame).

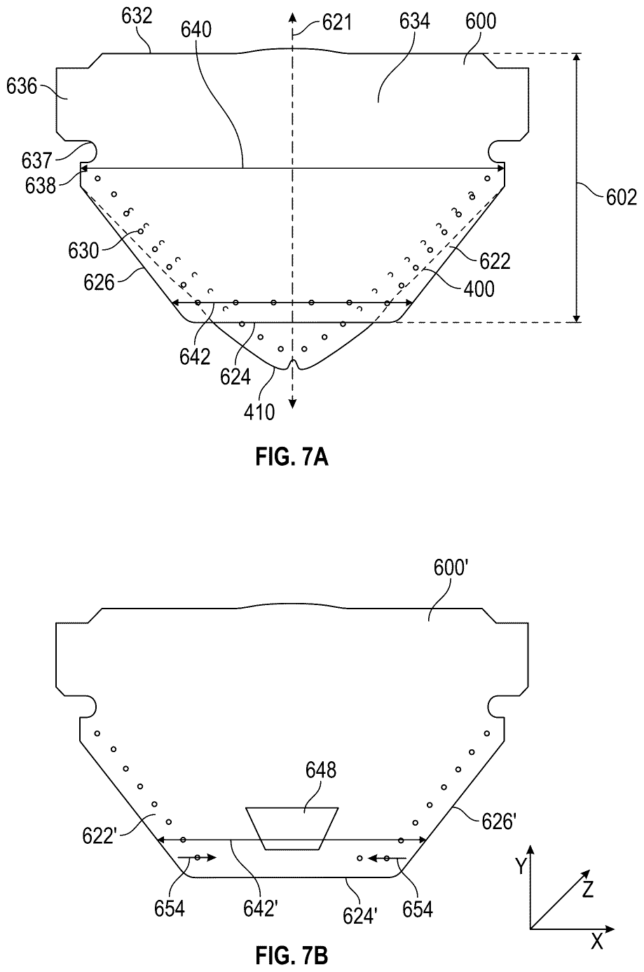

FIGS. 7A and 7B show a leaflet 600 in its as-cut or off-frame configuration and superimposed over the first leaflet 400 (shown in FIG. 7A) and in its compressed or on-frame configuration (represented by the leaflet 600′ in FIG. 7B).

The leaflet 600 may be similar to the leaflet 420 except it has a shorter axial length 602, which is defined in a direction of a central longitudinal axis 621 of the leaflet 600. As a result, the inflow edge 624 of its cusp edge portion 622 is offset from the inflow end 410 of the first leaflet 400, toward the outflow edge portion 632 of the leaflet 600, thereby making the leaflet 600 shorter than the first leaflet 400 (but having a same or similar width, at least along the outflow edge portion 632). In some examples, the leaflet 600 is about 2 mm shorter than the first leaflet 400.

For all the leaflets described herein, various widths of the leaflets can be defined in the lateral direction (or circumferential direction and/or x direction). As a representative example, a maximum width 640 of the leaflet 600 (not accounting for the commissure tabs) can, in some examples be defined as the distance from one end of the cusp edge portion 622 to the other end adjacent the tabs 638, as shown in FIG. 7A. This width 640 can be the same or similar to the other leaflets described herein, and in some examples the same or similar to the first leaflet 400.

In some examples, the leaflet 600 (and the other leaflets described herein) can have a first width 642 between the side edges 626 of the cusp edge portion 622 in its off-frame configuration (as shown in FIG. 7A), where the first width 642 is measured closer to the inflow edge 424. When the leaflet 600 is attached to the frame, as shown in FIG. 7B, the leaflet 600′ can have a second width 642′ that is smaller than the first width 642, due to the compression or translation of the side edges 626′ closer to one another when attached to the frame. It should be noted that FIG. 7B is schematic, and even though the second width 642′ may not appear shorter than the first width 642 shown in FIG. 7A, when attached to an actual prosthetic valve frame, the second width 642′ can be smaller.

The leaflet 600 comprises a main body 634 with the outflow edge portion 632 (which may be the same or similar to the outflow edge portion 432) and the cusp edge portion 622. The cusp edge portion 622 includes the inflow edge 624 and side edges 626 which angle from opposite sides of the inflow edge 624.

Apertures 630, which may be pre-formed in the leaflet 600, can be spaced apart along each of the side edges 626.

The leaflet 600 can comprise opposing commissure tabs 636.

In some examples, the leaflet 600 includes smaller tabs 638 that are axially offset, relative to the central longitudinal axis 621, from the commissure tabs 636 by indented regions 637.

Similar to the inflow edge 424 of the leaflet 420, the inflow edge 624 of the leaflet 600 is straight (or relatively straight, which may include a slight wave or non-straight portion but still have an overall straight or linear shape) and distances the side edges 626 farther away from one another than the cusp edge portion 408 of the first leaflet 400. In some examples, the inflow edge 624 can be longer that the inflow edges 424 and 524, thereby positioning the side edges 626 farther apart from one another.

Thus, similar to as described above for leaflet 420, when the leaflet 600 is coupled to the frame (e.g., frame 202), the side edges 626 are moved and/or compressed inward toward one another (in the x direction) to couple to struts 214 of the frame 202 along the attachment line of the frame. Since the leaflet 600 has the shorter axial length 602 (than the leaflets 420 and 500, for example), it may not need to be compressed in the y or longitudinal direction to offset the inflow edge 624 from the inflow end of the frame, as discussed above.

The leaflet 600′ is shown in its compressed configuration in FIG. 7B. The leaflet 600′ may only be compressed in the lateral (or x) direction, as shown schematically by the arrows 654. FIG. 7B shows the compressed leaflet material 648, which results in increased bulging of the leaflet material and/or increased concavity/convexity of the main body 634 of the leaflet 600′, as described above. In this way, the leaflet 600′ can have a more 3D shape, as compared to the leaflet 600 in its off-frame configuration and as compared to the first leaflet 400 in its on-frame configuration. In some examples, the leaflet 600′ can also have increased mobility at its inflow end (e.g., due to the inflow edge 624 being longer).

In some examples, the resulting lateral compression of the leaflet 600′ is in a range of 10-15%, about 10%, or about 15%.

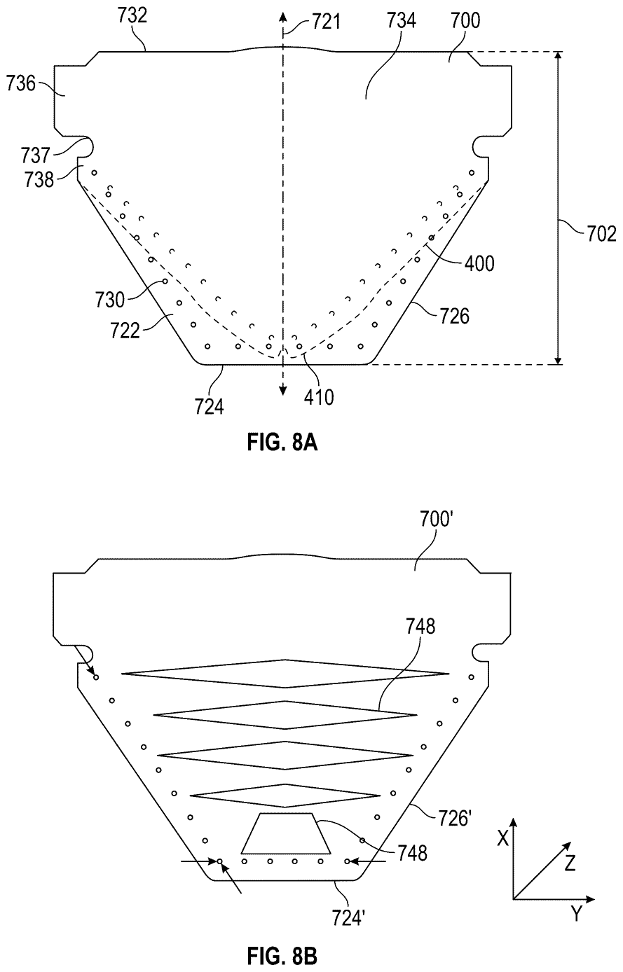

FIGS. 8A and 8B show a leaflet 700 in its as-cut or off-frame configuration and superimposed over the first leaflet 400 (shown in FIG. 8A) and in its compressed or on-frame configuration (represented by the leaflet 700′ shown in FIG. 8B).

The leaflet 700 may be similar to the leaflets 420, 500, and 600 (e.g., some combination thereof). For example, the leaflet 700 can have an axial length 702, which is defined in a direction of a central longitudinal axis 721 of the leaflet 700, that is slightly longer than the first leaflet 400 (e.g., somewhere between the axial length 502 of the leaflet 500 and the axial length of the leaflet 420). As a result, the inflow edge 724 of its cusp edge portion 722 is offset from (and extends past) the inflow end 410 of the first leaflet 400.

The same as or similar to the leaflets 420, 500, and 600, the leaflet 700 comprises a main body 734 with the outflow edge portion 732 (which may be the same or similar to the outflow edge portion 432) and the cusp edge portion 722. The cusp edge portion 722 includes the inflow edge 724 and side edges 726 which angle from opposite sides of the inflow edge 724.

Apertures 730, which may be pre-formed in the leaflet 700, can be spaced apart along each of the side edges 726.

The leaflet 700 can comprise opposing commissure tabs 736.

In some examples, the leaflet 700 includes smaller tabs 738 that are axially offset, relative to the central longitudinal axis 721, from the commissure tabs 736 by indented regions 737.

Similar to the inflow edge 424 of the leaflet 420, the inflow edge 724 of the leaflet 700 is straight (or relatively straight, which may include a slight wave or non-straight portion but still have an overall straight or linear shape) and distances the side edges 726 farther away from one another than the cusp edge portion 408 of the first leaflet 400. In some examples, the inflow edge 724 can be longer that the inflow edges 424 and 524 of leaflets 420 and 500, respectively, thereby positioning the side edges 726 farther apart from one another (similar to the side edges 626 of the leaflet 600).

Thus, when the leaflet 700 is coupled to the frame (e.g., frame 202), the side edges 726 are moved and/or compressed inward toward one another (in the x direction) to couple to struts 214 of the frame 202 along the attachment line of the frame, and the inflow edge 724 is compressed toward its outflow edge portion 732 to offset the inflow edge 724 from the inflow end 216 of the frame 202.

As a result, the leaflet 700′ is moved into its compressed configuration, as shown in FIG. 7B. FIG. 7B shows the compressed leaflet material 748, which results in increased bulging of the leaflet material and/or increased concavity/convexity of the main body 734′ of the leaflet 500′, as described above. In this way, the leaflet 700′ can have a more 3D shape, as compared to the leaflet 700 in its off-frame configuration and as compared to the first leaflet 400 in its on-frame configuration. In some examples, the leaflet 700′ can also have increased mobility at its inflow end (due to the longer length of the inflow edge 724).

In some examples, the resulting longitudinal or axial (y direction) and lateral (x direction) compression of the leaflet 700′ can be in a range of 10-25%.

In addition to providing the leaflets with a more 3D shape and improving leaflet mobility, the leaflet designs described above (e.g., for leaflets 420, 500, 600, and 700) can also provide optimal coaptation of the leaflets, such that during operation when included in a prosthetic heart valve implanted in vivo, the leaflets of the prosthetic heart valve fully close together (contact one another) at the level of their outflow edge portions (or free edges).

For example, the compression and/or outward buckling of the leaflets along their cusp edge portion (or scallop line), as described above for FIGS. 5A-8B, can result in lifting of the entire leaflet such that the angle of coaptation is smaller than a more traditional leaflet that is not compressed and/or does not have added leaflet material, such as the first leaflet 400. This results in improved coaptation, such that the leaflets of a prosthetic heart valve fully close at the center of coaptation, when implanted and operating in vivo.

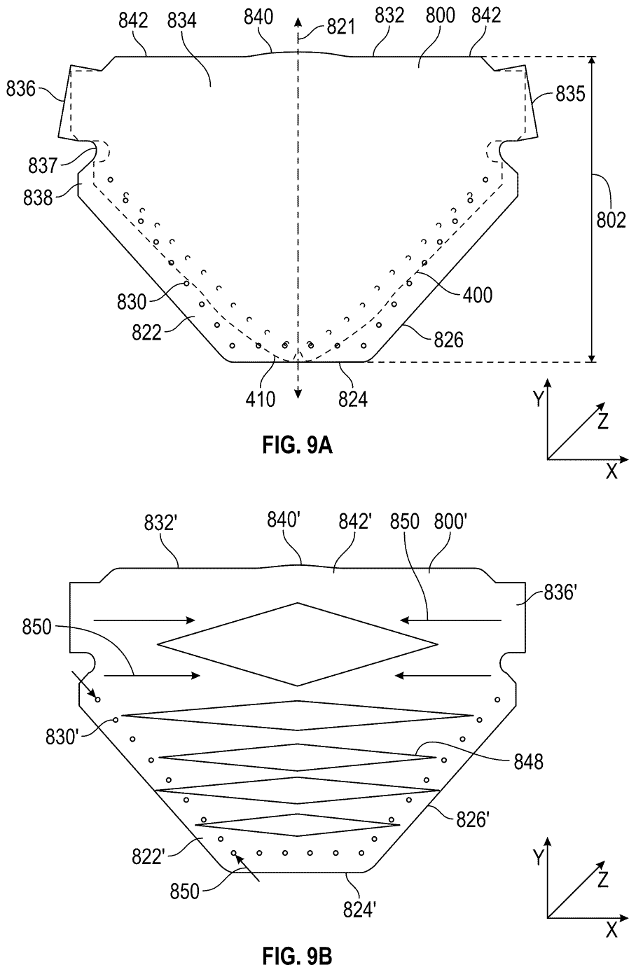

In some examples, to further improve coaptation between leaflets, the as-cut leaflet can be configured with added leaflet material at the level of the commissure. This can, in some examples, result in laterally distancing the commissure tabs farther apart from each other, as illustrated in the example of FIGS. 9A and 9B.

FIGS. 9A and 9B show a leaflet 800 in its as-cut or off-frame configuration and superimposed over the first leaflet 400 (shown in FIG. 9A) and in its compressed or on-frame configuration (shown in FIG. 9B).

The leaflet 900 may be similar to the leaflet 420 except it has added leaflet material at the level of the commissure tabs 836 and the commissure tabs 836 are angled relative to the straight portions of the outflow edge portion 832 and/or the inflow edge 824 (and the central longitudinal axis 821). For example, as shown in FIG. 8A, the outer edge 835 of each commissure tab 836 is angled at a non-perpendicular angle relative to the inflow edge 824 (and straight portions of the outflow edge portion 832) and at a non-zero angle relative to the central longitudinal axis 821. In this way, the outer edges 835 of the commissure tabs 836 are non-perpendicular to the inflow edge 824 and straight portions of the outflow edge portion 832. In contrast to the outer edge of the commissure tabs of the first leaflet 400, the outer edges 835 of the commissure tabs 836 angle laterally outward and upward toward the outflow edge portion 832. This can result in additional leaflet material at the commissure tabs 836.

The leaflet 900 can have a same or similar axial length 802 to that of the leaflet 420, where the axial length 802 is defined in the direction of the central longitudinal axis 821.

The leaflet 800 comprises a main body 834 with the outflow edge portion 832 (which may be the same or similar to the outflow edge portion 432) and the cusp edge portion 822. The cusp edge portion 822 includes the inflow edge 824 and side edges 826 which angle from opposite sides of the inflow edge 824 (the same or similar to side edges 426). Apertures 830 can be spaced apart along each of the side edges 826.

In some examples, the leaflet 800 includes smaller tabs 838 that are axially offset, relative to the central longitudinal axis 821, from the commissure tabs 836 by indented regions 837.

In some examples, a width (in the x or lateral direction) of the leaflet 800 at the level of the tabs 838 and/or the indented regions 837 can be larger than that of the first leaflet 400 and the leaflet 420. This may be due to the angling of the commissure tabs 836 causing the commissure tabs 836 to be spaced farther apart from one another in the lateral direction (x direction).

Similar to the inflow edge 424 of the leaflet 420, the inflow edge 824 of the leaflet 800 is straight (or relatively straight, which may include a slight wave or non-straight portion but still have an overall straight or linear shape) and distances the side edges 826 farther away from one another than the cusp edge portion 408 of the first leaflet 400.

Thus, when the leaflet 800 is coupled to the frame (e.g., frame 202), the side edges 826 are compressed inward toward one another (in the x direction) to couple to struts 214 of the frame 202 along the attachment line of the frame, and the inflow edge 824 is compressed toward its outflow edge portion 832 to offset the inflow edge 824 from the inflow end 216 of the frame 202. Additionally, due to the added material and angling at the commissure tabs 836, when being attached to the frame, the commissure tabs 836 can be similarly tucked, moved, and/or compressed away from the outflow edge portion such that they assume a straightened configuration (as shown in FIG. 9B, by commissure tabs 836′). In the compressed configuration, the outer edges 835′ of the commissure tabs 836′ can be parallel to (or closer to parallel to) the central longitudinal axis 821.

FIG. 9B shows the compressed leaflet material 848, which results in increased bulging or bowing outward of the leaflet material and/or increased concavity/convexity of the main body 834′ of the leaflet 800′, as described above. In this way, the leaflet 800′ can have a more 3D shape, as compared to the leaflet 800 in its off-frame configuration and as compared to the first leaflet 400 in its on-frame configuration.

The compression of the leaflet 800′ that occurs when attaching to the frame can result in the apertures 830′ moving toward one another, the outer edge 835′ of the commissure tabs 836′ moving toward one another, and the region of the indented regions 837′ moving toward one another (in the x or lateral direction). This is denoted schematically in FIG. 9B by arrows 850.

In this way, the angling of the commissure tabs 836 in the as-cut configuration of the leaflet 800 results in added leaflet material at the commissural level, thereby improving coaptation and contact between the leaflets 800′ when mounted in a prosthetic heart valve and implanted in vivo.

In some examples, the outflow edge portion (or free edge) of any of the leaflets described herein (e.g., any of leaflets 420, 500, 600, 700, or 800) can include a central bump or protrusion that extends axially outward and away from straight portions of the outflow edge portion. Using the leaflet 800 of FIGS. 9A and 9B as an example, the outflow edge portion 832 includes a central protrusion 840 that extends outward and away from straight portions 842 of the outflow edge portion 832, in the axial direction (or in a direction of the y axis). In this way, the outflow edge portion 832 can include a straight portion 842 on each side of the central protrusion 840, with each straight portion 842 extending to a respective commissure tab 836. As explained further below with reference to FIGS. 12, 13, and 15A-17, the central protrusion 840 can improve the contact between leaflets in a prosthetic heart valve in the closed state (e.g., during diastole when implanted in a patient). For example, the central protrusion 840 can reduce or eliminate a central gap between the outflow edge portions of the leaflets of a prosthetic heart valve when in the closed state.

FIG. 10 shows the leaflet 500 (from FIG. 6A) alone and FIG. 11 shows a portion of an exemplary prosthetic valve with leaflets 500 attached to the frame 202. The dashed lines 232, 234 in FIGS. 10 and 11 show the corresponding locations of the leaflet 500 off the frame (FIG. 10) and when mounted inside the frame (FIG. 11). FIG. 11 shows the leaflets 500′ attached to an inner skirt 236 (e.g., with stitches 242), which is attached, in turn, to the struts 214 of the frame 202 with stitches 240, for example.

It should be noted that the inner skirt 236 is depicted in FIG. 11 with excess skirt material extending below the inflow end 216 of the frame 202. In some examples, this excess skirt material can be wrapped around the inflow apices 208 of the frame 202 such that a portion of the inner skirt 236 covers a portion of the outer surface of the frame 202. In some examples, this excess skirt material can be trimmed at the inflow end 216 of the frame 202.

In some examples, the leaflets 500′ can be attached directly to the struts 214 of the frame 202 without the inner skirt 236.

As shown in FIG. 11, each commissure tab 536′ of each leaflet 500′ is paired with a commissure tab 536′ of an adjacent leaflet 500′ to form a pair of commissure tabs that extends through a commissure window 222 in the frame 202 and is attached thereto to form a commissure.

As introduced above, in some examples, the length of the inflow edge 524 can be determined based on a distance between two lowermost non-apical junctions 230 at or adjacent to the inflow end 216 of the frame, on both sides of an inflow apex 208 (or apex region) of the frame 202. For example, as shown in FIG. 11, each angled strut 214 at the inflow end 216 extends between a respective inflow apex 208 and non-apical junction 230. The non-apical junctions 230 adjacent to the inflow end 216 are axially offset from the inflow apices 208.

In some examples, the inflow edge 524′ can be coupled to the frame 202 at a level of or adjacent to the non-apical junctions 230, between the two non-apical junctions 230.

As such, when the inflow edge 524′ is compressed and offset away from the inflow apices 208 at the inflow end 216 and towards the outflow end 218 of the frame 202, the inflow edge 524′ can be offset from the inflow apices 208 of the frame 202, toward the outflow end of the frame 202, by an axial distance 238. In some examples, the axial distance 238 is a range of 1 to 3 mm, 1.5 to 2.5 mm, or about 2 mm.

The compression of the leaflet 500′ due to offsetting the inflow edge 524′ from the inflow end 216 of the frame 202 and moving the side edges 526′ toward one another to couple them to the struts 214 following the scallop line (as shown FIG. 11, see the struts 214 extending from one commissure window 222 to the inflow end 216 and then to an adjacent commissure window 222) can create a relatively smooth concave/convex zone (depending on which side of the leaflet you are viewing) extending along the leaflet's belly (e.g., the zone of the main body 534′ between the dashed lines 232 and 234 in FIG. 11).

In this way, by offsetting the inflow edges 524′ of the leaflets 500′ away from the inflow end 216 of the frame, tissue overgrowth along the inflow ends of the leaflets can be reduced. Further, the trapezoidal-shaped cusp edge portion with added leaflet material for compressing and/or bowing leaflet material of the leaflet when coupling the cusp edge portion to the scallop line of the frame can give the leaflet a 3D surface geometry when attached to the frame that is more like a native leaflet. As a result, the leaflets can have improved mobility and coaptation, along with improving washout of blood that can accumulate between the leaflets and skirt.

FIG. 12 shows an exemplary leaflet 900 with a free or outflow edge portion 904 comprising a bump or protrusion 910 that provides the leaflet 900 with excess leaflet material that can enable the leaflet to seal or close against adjacent leaflets in a prosthetic valve during operation when implanted in a patient (e.g., during diastole). The leaflet 900 comprises a main body 902 with the outflow edge portion 904 and a cusp edge portion 906. The leaflet 900 also comprises opposing commissure tabs 908 disposed on opposite sides of the main body 902.

In some examples, the outflow edge portion 904 can be continuous with outflow edges 912 of the commissure tabs 908.

The cusp edge portion 906 is shown in FIG. 12 as having a curved shape at its inflow end. However, in some examples, the cusp edge portion 906 can have a shape similar to any of the other leaflets described herein, such as one of the trapezoidal-shaped cusp edge portions of leaflets 420, 500, 600, 700, or 800.

The cusp edge portion 906 includes the protrusion 910. The protrusion 910 can be centered along the central longitudinal axis 916 of the leaflet 900, and thus can be referred to herein as the central protrusion 910.

In some examples, the protrusion 910 (and/or the protrusion or bump of other leaflets described herein) can be offset slightly from the central longitudinal axis 916.

In some examples, such as the example shown in FIG. 12, the cusp edge portion 906 can include straight portions 914 on both sides of the protrusion 910. For example, the protrusion 910 can be disposed between the two straight portions 914 of the leaflet 900. As such, the protrusion 910 extends axially outward and away from the straight portions 914 of the leaflet 900.

A width 918 and height 924 of the protrusion 910 can be sized to provide adequate closure in a desired range of valve working expansion diameters of a prosthetic heart valve in which it is implanted.

For example, FIG. 13 is a schematic top view of a prosthetic valve 920 that comprises a frame 922 and a plurality of leaflets 900 (e.g., three, however other numbers of leaflets 900 are possible such as two) mounted inside the frame. During operation of the prosthetic valve 920, when implanted in a patient, the leaflets 900 open and close (e.g., during systole and diastole, respectively). The leaflets 900 are shown in a closed position in FIG. 13. As show in FIG. 13, the central protrusions 910 of the leaflets 900 allow the leaflets to fully close (or coapt) against one another (and in some examples, seal against one another). As a result, backflow during diastole can be reduced or prevented.

In some examples, a prosthetic valve can be designed for expansion (radial expansion to an expanded configuration) to a working expansion diameter within a range of working expansion diameters. As such, the open and closed states of the leaflets in the prosthetic valve can vary from their optimal configurations when the prosthetic valve is expanded beyond or below its nominal (or preset) working expansion diameter. For example, two prosthetic valves can include the same sized leaflets, but be expanded to different working expansion diameters when implanted. As a result, the leaflets 930 in a prosthetic valve 932 with the larger working expansion diameter may not fully coapt (seal or close together) in the closed state, thereby resulting in a central opening 934 through which blood can regurgitate, as shown in FIG. 14.

Thus, the width 918 and height 924 of the protrusion 910 of the leaflets 900 of the prosthetic valve 920 can be sized such that the leaflets 900 fully coapt or seal/close together in the closed position (e.g., during diastole), for a range of working expansion diameters. For example, the protrusion 910 can be sized such that the leaflets 900 of the prosthetic valve 920 can close together without a gap or central opening therebetween when the prosthetic valve is radially expanded to a working expansion diameter in a range of 26-34 mm. As a result, the leaflets 900 can be used in prosthetic valves having various working expansion diameters, or in valves configured to expand to a working expansion diameter within a range of working expansion diameters.

Further, the protrusion 910 can be utilized within the leaflet 900 without changing an overall shape of the leaflet 900 (such as a size of its main body 902 or cusp edge portion 906).

FIGS. 15A-16 show exemplary leaflets that include a central protrusion, similar to the protrusion 910 of leaflet 900, as well as depressed regions on both sides of the central protrusion that are depressed inward into the main body of the leaflets relative to the central protrusion.

Turning first to FIGS. 15A and 15B, a leaflet 950 comprises a main body 952 with an outflow edge portion 954 (or free edge portion) and a cusp edge portion 956. The leaflet 950 can also include opposing commissure tabs 958 disposed on opposite sides of the main body 952.

The outflow edge portion 954 includes a protrusion 960 (which can be aligned with a central longitudinal axis 966 of the leaflet 950 in some examples and be referred to herein a central protrusion 960) and depressed regions 962 (or recessed regions or portions), with the protrusion 960 disposed between the depressed regions 962.

Each depressed region 962 can extend between a respective side of the protrusion 960 and a respective commissure tab 958.

The depressed regions 962 are depressed inward into the main body 952 of the leaflet 950 relative to the protrusion 960. This configuration can provide the leaflet 950 with more material along is outflow edge portion 954 than the leaflet 900 of FIG. 12, for example. Said another way, if straightened out, the outflow edge portion 954 of the leaflet 950 would be longer than the outflow edge portion 904 of leaflet 900. This extra arc length or material of the outflow edge portion 954 can allow the outflow edge portion 954 to stretch further under pressure (e.g., diastole), thereby facilitating full closure of the leaflets of a prosthetic valve.

Thus, by having leaflets with the outflow edge portion 954 with the protrusion 960 and depressed regions 962, a prosthetic valve can be expanded to larger working expansion diameter, while allowing the leaflets to fully close during diastole and open during systole without contacting the frame.

FIG. 15A shows an example of the leaflet 950 where the protrusion 960 is aligned with outflow edges 964 of the commissure tabs 958 (as shown schematically by the dashed line 968). The depressed regions 962 depress inwards into the main body 952 relative to (or from) the outflow edges 964 of the commissure tabs 958.

FIG. 15B shows an example of the leaflet 950 where the protrusion 960 extends axially outward and away from the outflow edges 964 of the commissure tabs 958 (and the dashed line 968).

In some examples, this shape of the outflow edge portion 954 can more closely resemble that of a native leaflet.

Similar to the leaflet 900, the width and height of the protrusion 960 can be sized to provide closure of the leaflets 900 mounted in a prosthetic heart valve, for a desired range of valve working expansion diameters of the prosthetic heart valve, when implanted.

FIG. 16 shows an exemplary leaflet 1000 comprising a main body 1002 with an outflow edge portion 1004 (or free edge portion) and a cusp edge portion 1006. The leaflet 1000 can also include opposing commissure tabs 1008 disposed on opposite sides of the main body 1002.

The outflow edge portion 1004 includes a protrusion 1010. In some examples, the protrusion 1010 can be referred to as a central protrusion 1010. In some examples, the central protrusion can be aligned with a central longitudinal axis 1016 of the leaflet 1000.

The outflow edge portion 1004 includes depressed regions 1012 (or recessed regions or portions), with the protrusion 1010 disposed between the depressed regions 1012. The depressed regions 1012 are depressed inward into the main body 1002 from the protrusion 1010 and ends 1014 of the outflow edge portion 1004 that are disposed adjacent to the commissure tabs 1008.

In some examples, the ends 1014 of the outflow edge portion 1004 are tabs or portions of the commissure tabs 1008 (such as outflow edges of the commissure tabs 1008).

Each depressed region 1012 can extend between a respective side of the protrusion 1010 and a respective end 1014 of the outflow edge portion 1004.

In some examples, as shown in FIG. 16, the commissure tabs 1008 are angled relative to the central longitudinal axis 1016 of the leaflet 1000. For example, an outer edge 1018 of each commissure tab 1008 can be non-parallel to the central longitudinal axis 1016. In some examples, the outer edge 1018 can be angled away from the outflow edge portion 1004 and toward the cusp edge portion 1006 at a non-zero angle relative to the central longitudinal axis 1016. This can cause an outflow edge 1020 of the commissure tab 1008 to be non-perpendicular to the central longitudinal axis 1016.

In some examples, as shown in FIG. 16, the angling of the commissure tabs 1008 can result in the outer edges 1018 being arranged in-line with the curved cusp edge portion 1006 (or at least a portion of the cusp edge portion 1006 that is adjacent to the commissure tabs 1008).

In some embodiments, the protrusion (or central protrusion) of any of the leaflets described herein (e.g., the protrusion 840, 910, 960, 1010, or 1060) can be thicker (in the z direction) than other portions of the leaflet. For example, when the leaflet is made of a polymeric material or materials, the leaflet can have different thicknesses at different regions of the leaflet.

A thicker protrusion, or central protrusion, can reduce a likelihood of abrasion due to contact with adjacent leaflets and/or the valve frame. As a result, a longevity of the leaflets can be increased.

In some examples, portions of the outflow edge portion disposed between the commissures and the protrusion (e.g., the straight portions 914 or depressed regions 962, 1012, or 1064) can be thinner than the protrusion and/or a remainder of the leaflet. This may be possible since these portions of the leaflet undergo little to no stress. These thinner leaflet portions can further enable better mobility and coaptation to increase stability of the leaflet during closing of the leaflets.

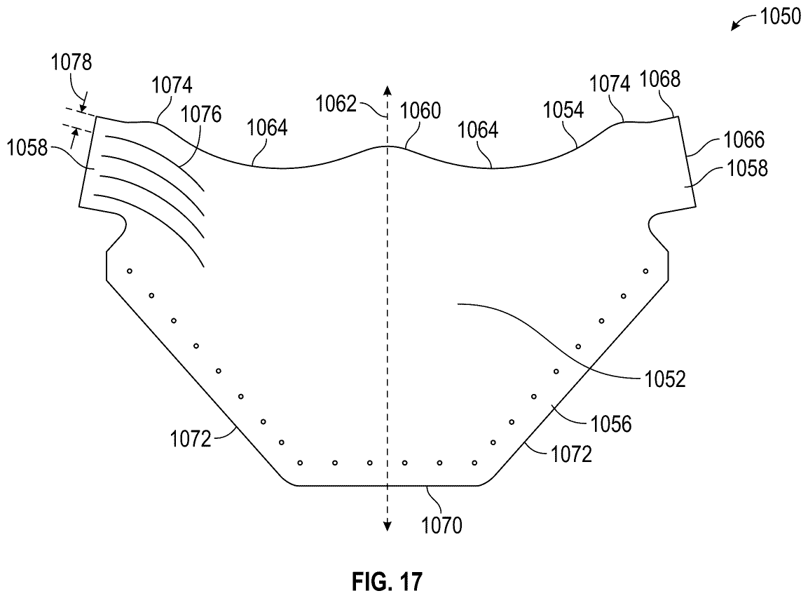

FIG. 17 shows an exemplary leaflet 1050 including geometrical features configured to improve coaptation when included in an implanted prosthetic heart valve. The leaflet 1050 can be shaped such that its outflow edge portion (its coaptation region) is lower or disposed farther away from the outflow end of the prosthetic valve in which it is attached.

As shown in FIG. 17, the leaflet 1050 comprises a main body 1052 with an outflow edge portion 1054 (or free edge portion) and a cusp edge portion 1056. The leaflet 1050 can also include opposing commissure tabs 1058 disposed on opposite sides of the main body 1002.

In some examples, as shown in FIG. 17, the commissure tabs 1058 are angled upwards (in the view of FIG. 17), toward the outflow edge portion 1054. For example, the commissure tabs 1058 are angled relative to a central longitudinal axis 1062 of the leaflet 1050 such that an outer edge 1066 of each commissure tab 1058 is non-parallel to the central longitudinal axis 1062. The outer edge 1066 is angled toward the central longitudinal axis 1062 and the outflow edge portion 1054 at a non-zero angle relative to the central longitudinal axis 1062. As such, an outflow edge 1068 of the commissure tab 1058 can be non-perpendicular to the central longitudinal axis 1062.

Similar to the leaflet 800 described above, when attaching the leaflet 1050 to a frame of a prosthetic valve (such as the frame 202), the commissure tabs 1058 are bent downwards, away from the outflow edge portion 1054. In some examples, this bending can result in the outer edges 1066 being parallel or close to parallel to the central longitudinal axis 1062. This movement of the commissure tabs 1058 when attaching to the frame can lift the outflow end (or outflow edge portion 1054) of the leaflet 1050 upwards (toward an outflow end of the frame) and result in added leaflet material at the region adjacent to the outflow edge portion 1054 (similar to as described above for the leaflet 800). This can, in some examples, improve coaptation depth between the leaflets 1050 of the prosthetic valve (such that they fully close together).

In some examples, the cusp edge portion 1056 can have a trapezoidal shape with edges (including an inflow edge 1070 and side edges 1072) that are configured to be compressed and/or moved toward one another when coupling to the frame along the scallop line, as described above with reference to FIGS. 5A-11.

The outflow edge portion 1054 includes a central protrusion 1060 (which can, in some examples, be aligned with a central longitudinal axis 1062 of the leaflet 1050) and depressed regions 1064 (or recessed regions or portions), with the central protrusion 1060 disposed between the depressed regions 1064.

The central protrusion 1060 can be similar to those of the leaflets shown in FIGS. 15A-16, except the central protrusion 1060 can be spaced farther away from the outflow edges 1068 of the commissure tabs 1058. This can be achieved by forming deeper depressed regions 1064. For example, the depressed regions 1064 are depressed inward into the main body 1052 from the central protrusion 1060 and the outflow edges 1068 of the commissure tabs 1058.

In some examples, the depressed regions 1064 can improve coronary access through the frame of the implanted prosthetic valve including the leaflets 1050 for certain interventional procedures (such as valve-in-valve procedures).

In some examples, the leaflet 1050 can include additional bumps or protrusions, referred to herein as tab bumps 1074. Each tab bump 1074 is disposed between a respective commissure tab 1058 and depressed region 1064. The tab bumps 1074 can be configured to distance tension lines 1076 of the leaflet 1050 farther away from the edge of the leaflet 1050 (as shown by distance 1078 in FIG. 17).

This can prevent stress concentrations from occurring near or at the outflow edge of the outflow edge portion 1054. Instead, as shown schematically in FIG. 17, the stress or tension lines 1076 can be maintained within the contour of the tissue material of the leaflet 1050 and away from the tab bumps 1074.

In some examples, as shown in FIG. 17, the peak of the central protrusion 1060 can be lower or offset toward the inflow edge 1070 relative to the tab bumps 1074.

In some examples, the peak of the central protrusion 1060 may not be lower than or offset from the tab bumps 1074.

In some examples, it may be desirable to have a three-dimensional leaflet with a rounded cusp edge or cusp edge portion when assembled to a prosthetic valve frame (which can be referred to as the 3D assembled configuration). Having a rounded cusp edge in the 3D assembled configuration can, in some examples, improve washout of blood during diastole and reduce the likelihood of low-flow or stagnant regions in the prosthetic valve.

FIG. 18 shows an exemplary leaflet 1100 with a rounded or circular cusp edge 1102. In some examples, the angle of curvature of the cusp edge 1102 can be in a range of 100-140 degrees, 110-130 degrees, 115-125 degrees, or about 120 degrees. In this way, when the angle of curvature is about 120 degrees, the cusp edge 1102 can be referred to as a circular cusp edge 1102.

While the leaflet 1100 can be cut to form the rounded or circular cusp edge 1102 in 2D (e.g., when held in a flattened configuration on a flat surface, or in a flat plane, as shown in FIG. 18), when assembled in and to an annular frame of a prosthetic valve (such as the frame 202), the two-dimensional circular cusp edge 1102 is no longer circular, but instead distorted due to the 3D shape assumed by the leaflet 1100 after attachment.

To create a 3D leaflet with a rounded or circular cusp edge when mounted within and to an annular frame of a prosthetic valve, a 3D leaflet can be created virtually (such as by using computer aided design software) and then used to form a 2D leaflet cutting template for cutting a real leaflet in a flattened or flat configuration.

For example, FIGS. 19A-21 depict a method for forming a 3D leaflet with a rounded or circular cusp edge when mounted within a prosthetic valve frame. The method can include forming a partial first cylinder (e.g., ⅓ of a cylinder) to represent a leaflet (and thus the first cylinder can be referred to as a “leaflet cylinder”). The method further includes intersecting and cutting the first cylinder with a second cylinder (e.g., a full cylinder that can be referred to as a “cutting cylinder”). As a result of this cutting, a 3D curved leaflet shape with a rounded or circular cusp edge is formed. This leaflet shape can then be transformed into a 2D shape (e.g., laid flat within a 2D plane) which is then used as a leaflet template for cutting a leaflet material into a leaflet. When attaching the cut leaflet (which was cut in the flattened state) to an annular prosthetic valve frame, the leaflet can then assume the 3D curved leaflet shape (which was originally formed by cutting the first cylinder with a second cylinder). In this way, a portion of a cusp edge portion (or cusp edge) of a leaflet can have a shape of a first cylinder intersecting with a second cylinder and the main body of the leaflet can have a concave (and/or outward bulging) shape when attached to an annular frame of a prosthetic heart valve.

In some examples, as shown in FIGS. 19A-19C (which show front, perspective, and side views, respectively), the method can include cutting (or intersecting) a leaflet cylinder 1110 with a cutting cylinder 1112. In FIGS. 19A-19C, the cutting cylinder 1112 and the leaflet cylinder 1110 are perpendicular to one another. Said another way, to cut the leaflet cylinder 1110, the cutting cylinder 1112 intersects with the leaflet cylinder 1110 at a 90-degree angle. A vertical line is shown adjacent to one end of the cutting cylinder 1112 for reference in FIG. 19C.

The cylindrical shape of the leaflet cylinder 1110 (or ⅓ of a cylinder shape), which represents the leaflet, can be chosen according to a desired assembled size of the leaflet. For example, it may be desired for the leaflet to have a specified width which is based on an expanded diameter of the prosthetic valve frame. As such, a diameter of the leaflet cylinder 1110 can be specified based on the desired width of the final leaflet and/or the diameter of the prosthetic valve frame (such as the frame's inner diameter at its lower or inflow end).

As an example, for a 23 mm prosthetic valve (or valve frame) including three leaflets, the leaflet cylinder 1110 can have a diameter in a range of 21.8-22.5 mm, about 22 mm, or 22.1 mm.

In some examples, the cutting shown in FIGS. 19A-19C can be performed in a computer aided design (CAD) software, resulting in a 3D cut leaflet shape 1114, as shown in FIG. 20. The 3D cut leaflet shape 1114 can be flattened into a 2D cut leaflet shape 1116 in order to derive the two-dimensional cutting pattern (or template or die) that can be used to cut a chosen leaflet material in a flattened configuration. When the cut leaflet is attached to the annular frame or the prosthetic valve, it will re-assume the 3D shape of the 3D cut leaflet shape 1114. As such, a die or template for cutting a leaflet, which results in a 3D leaflet with a rounded or circular cusp edge when attached to a prosthetic valve frame can be more easily and precisely achieved.

In some examples, the cutting cylinder can be angled relative to the leaflet cylinder at a different angle than 90 degrees (or at an angle relative to perpendicular). For example, as shown in FIG. 21, the cutting cylinder 1112 can intersect the leaflet cylinder 1110 at a non-perpendicular angle. In some examples, the non-perpendicular angle shown in FIG. 21 is 10 degrees relative to a vertical axis or central longitudinal axis 1118 of the leaflet

### Cylinder 1110.

In some examples, the non-perpendicular angle for intersecting and cutting the leaflet cylinder 1110 with the cutting cylinder 1112 can be more or less than 10 degrees relative to the central longitudinal axis 1118, such as in a range of 1-30 degrees (and/or including 30 degrees). The larger angle, beyond 90 degrees or the central longitudinal axis 1118, can result in a 2D cut leaflet shape 1120 having a cusp edge 1122 with a more parabolic shape and longer axial length 1124, as compared to the cusp edge 1115 and axial length 1117 of the 2D cut leaflet shape 1116, and the 3D cut leaflet shape 1114. This comparison is depicted in FIG. 22 with the 2D cut leaflet shape 1116 superimposed over the 2D cut leaflet shape 1120. However, both cut leaflet shapes can have a smooth, curved shape for their cusp edge (without any sharp edges or breaks in the curve). For example, the resulting leaflets can have a continuously curved rounded cusp edge.

As the non-perpendicular angle of the cutting cylinder 1112 increases, an arc length of the 2D cut leaflet shape, and thus the 3D cut leaflet shape, increases (as shown in the example of FIG. 22).

The cutting angle can be chosen to improve various functional characteristics of the leaflet, such as improved leaflet coaptation and/or improved washout from the prosthetic valve when implanted in a patient. In some examples, the greater cutting angles which result in the increased axial length of the leaflet can increase the mobility of the leaflet when attached to the frame and when the prosthetic valve is implanted and operating in a patient.

FIG. 23 shows a 3D leaflet 1130 resulting from cutting leaflet material with the 2D cut leaflet shape 1116 of FIG. 20, mounted inside the frame 202. As shown in FIG. 23, the leaflet 1130 has a round or circular cusp edge 1132 having the shape of a first cylinder intersecting with a second cylinder at a perpendicular angle.

In some examples, at least an inflow end portion of the cusp edge 1132 has the rounded or circular shape. In some examples, all or a majority of the cusp edge 1132 has the rounded or circular shape.

A main body 1134 of the leaflet 1130 can have a concave shape, or a portion that bulges, buckles, or otherwise pops outward relative to the cusp edge 1132 (which will be coupled to the frame), when mounted inside and/or coupled to the frame 202. In some examples, the inflow end portion of the main body 1134, adjacent to the cusp edge 1132, can be sphere-shaped.

In some examples, the leaflet 1130 can have opposing commissure tabs that are secured to commissure windows 222. The commissure tabs of the leaflet 1130 can be the same or similar to any of the commissure tabs of the leaflets described herein.

As shown in FIG. 23, an inflow end 1136 of the cusp edge 1132 of the leaflet 1130 is spaced away (axially away) from the inflow end 216 of the frame 202.

FIGS. 24A and 24B show a leaflet 1140 resulting from cutting leaflet material with the 2D cut leaflet shape 1120 of FIG. 22, mounted inside the frame 202. As shown in FIGS. 24A-24B, the leaflet 1140 has a round or circular cusp edge 1142 having the shape of a first cylinder intersecting with a second cylinder at a non-perpendicular angle.

In some examples, at least an inflow end portion of the cusp edge 1142 has the rounded or circular shape. In some examples, all or a majority of the cusp edge 1142 has the rounded or circular shape.

A main body 1144 of the leaflet 1140 can have a concave shape, or a portion that bulges, buckles, or otherwise pops outward relative to the cusp edge 1142 (which will be coupled to the frame), when mounted inside and/or coupled to the frame 202. In some examples, as shown in FIG. 24B, the inflow end portion of the main body 1144, adjacent to the cusp edge 1142, can be sphere-shaped.

In some examples, the leaflet 1140 can have opposing commissure tabs that are secured to commissure windows 222. The commissure tabs of the leaflet 1140 can be the same or similar to any of the commissure tabs of the leaflets described herein.

As shown in FIG. 24A, an inflow end 1146 of the cusp edge 1142 of the leaflet 1140 is spaced closer to the inflow end 216 of the frame 202 than the cusp edge 1132 of the leaflet 1130. This can be due to the longer axial length of the leaflet 1140 than the leaflet 1130.

In some examples, the inflow end 1146 of the cusp edge 1142 can be offset from the inflow end 216 of the frame 202.

In some examples, the leaflets 1130 and/or leaflets 1140 can be coupled to the frame 202 (or another frame) such that portions of their cusp edge are moved inward, toward a center of the main body of the leaflets when coupling the cusp edge to the frame (the same or similar to as described above for the leaflets of FIGS. 4-11). As a result, the leaflet material of the main body can be compressed and/or buckle or bow outwards to provide the main body with an even more concave (on one side of the leaflet) and 3D shape.

In some examples, the leaflet 1130 and/or the leaflet 1140 can comprises one or more features of the additional leaflets described herein. For example, the leaflet 1130 and/or the leaflet 1140 can comprise opposing commissure tabs that angled inwards, toward one another (such as in the leaflet 800 of FIG. 9A or leaflet 1050 of FIG. 17) or outwards, away from one another (such as in the leaflet 1000 of FIG. 16). As another example, the leaflet 1130 and/or the leaflet 1140 can comprise a central protrusion or bump, such as in the leaflets 420, 500, 600, 700, 800, 900, 950, 1000, or 1050.

FIGS. 25A-26B show another method in which the cutting cylinder 1112 is passed through an existing (previously cut) leaflet 1150 with a non-rounded cusp edge (only a portion of which is shown in FIGS. 25A-26B for ease of illustration). This can result in a leaflet 1150′ with a cusp edge 1152′ having a rounded, or continuously curved shape. In some examples, the radii of curvature (determined by the cutting cylinder 1112) of the cusp edge 1152′, as-cut, can be preserved when coupling the leaflet to the frame of the prosthetic valve.

This method may be performed with the perpendicularly extending cutting cylinder 1112, and can be helpful if modifying existing, or pre-cut, leaflets. The existing leaflet can be cut by the cutting cylinder 1112 either when provided (or held) in a 3D state (as shown in FIGS. 26A and 26B), or in its flattened 2D configuration (as shown in FIGS. 25A and 25B).

The leaflets described herein can comprise pericardial tissue (for example, bovine pericardial tissue), synthetic materials, or various other suitable natural or synthetic materials as known in the art and described in U.S. Pat. No. 6,730,118, which is incorporated by reference herein. In some examples, the leaflets described herein can comprise a biocompatible polymeric material.

### Delivery Techniques

For implanting a prosthetic valve within the native aortic valve via a transfemoral delivery approach, the prosthetic valve is mounted in a radially compressed state along the distal end portion of a delivery apparatus. The prosthetic valve and the distal end portion of the delivery apparatus are inserted into a femoral artery and are advanced into and through the descending aorta, around the aortic arch, and through the ascending aorta. The prosthetic valve is positioned within the native aortic valve and radially expanded (e.g., by inflating a balloon, actuating one or more actuators of the delivery apparatus, or deploying the prosthetic valve from a sheath to allow the prosthetic valve to self-expand). Additionally and/or alternatively, a prosthetic valve can be implanted within the native aortic valve in a transapical procedure, whereby the prosthetic valve (on the distal end portion of the delivery apparatus) is introduced into the left ventricle through a surgical opening in the chest and the apex of the heart and the prosthetic valve is positioned within the native aortic valve. Additionally and/or alternatively, in a transaortic procedure, a prosthetic valve (on the distal end portion of the delivery apparatus) is introduced into the aorta through a surgical incision in the ascending aorta, such as through a partial J-sternotomy or right parasternal mini-thoracotomy, and then advanced through the ascending aorta toward the native aortic valve.

For implanting a prosthetic valve within the native mitral valve via a transseptal delivery approach, the prosthetic valve is mounted in a radially compressed state along the distal end portion of a delivery apparatus. The prosthetic valve and the distal end portion of the delivery apparatus are inserted into a femoral vein and are advanced into and through the inferior vena cava, into the right atrium, across the atrial septum (through a puncture made in the atrial septum), into the left atrium, and toward the native mitral valve. Additionally and/or alternatively, a prosthetic valve can be implanted within the native mitral valve in a transapical procedure, whereby the prosthetic valve (on the distal end portion of the delivery apparatus) is introduced into the left ventricle through a surgical opening in the chest and the apex of the heart and the prosthetic valve is positioned within the native mitral valve.

For implanting a prosthetic valve within the native tricuspid valve, the prosthetic valve is mounted in a radially compressed state along the distal end portion of a delivery apparatus. The prosthetic valve and the distal end portion of the delivery apparatus are inserted into a femoral vein and are advanced into and through the inferior vena cava, and into the right atrium, and the prosthetic valve is positioned within the native tricuspid valve. A similar approach can be used for implanting the prosthetic valve within the native pulmonary valve or the pulmonary artery, except that the prosthetic valve is advanced through the native tricuspid valve into the right ventricle and toward the pulmonary valve/pulmonary artery.

Another delivery approach is a transatrial approach whereby a prosthetic valve (on the distal end portion of the delivery apparatus) is inserted through an incision in the chest and an incision made through an atrial wall (of the right or left atrium) for accessing any of the native heart valves. Atrial delivery can also be made intravascularly, such as from a pulmonary vein. Still another delivery approach is a transventricular approach whereby a prosthetic valve (on the distal end portion of the delivery apparatus) is inserted through an incision in the chest and an incision made through the wall of the right ventricle (typically at or near the base of the heart) for implanting the prosthetic valve within the native tricuspid valve, the native pulmonary valve, or the pulmonary artery.

In all delivery approaches, the delivery apparatus can be advanced over a guidewire previously inserted into a patient's vasculature. Moreover, the disclosed delivery approaches are not intended to be limited. Any of the prosthetic valves disclosed herein can be implanted using any of various delivery procedures and delivery devices known in the art.

Any of the systems, devices, apparatuses, etc. herein can be sterilized (for example, with heat/thermal, pressure, steam, radiation, and/or chemicals, etc.) to ensure they are safe for use with patients, and any of the methods herein can include sterilization of the associated system, device, apparatus, etc. as one of the steps of the method. Examples of heat/thermal sterilization include steam sterilization and autoclaving. Examples of radiation for use in sterilization include, without limitation, gamma radiation, ultra-violet radiation, and electron beam. Examples of chemicals for use in sterilization include, without limitation, ethylene oxide, hydrogen peroxide, peracetic acid, formaldehyde, and glutaraldehyde. Sterilization with hydrogen peroxide may be accomplished using hydrogen peroxide plasma, for example.

### Additional Examples of the Disclosed Technology

In view of the above-described implementations of the disclosed subject matter, this application discloses the additional examples enumerated below. It should be noted that one feature of an example in isolation or more than one feature of the example taken in combination and, optionally, in combination with one or more features of one or more further examples are further examples also falling within the disclosure of this application.

Example 1. We claim a prosthetic heart valve comprising: a frame that is radially expandable from a radially collapsed configuration to a radially expanded configuration, wherein the frame has an inflow end and an outflow end; and a plurality of leaflets mounted on an inside the frame, wherein each leaflet comprises a main body with a cusp edge portion and an outflow edge portion, wherein each leaflet is compressed in an X direction and/or Y direction to form a concave shape.

Example 2. The prosthetic heart valve of any example herein, particularly example 1, wherein the cusp edge portion has a trapezoidal shape with two angled side edges and an inflow edge extending between the two angled side edges, and wherein the inflow edge defines an inflow end of the cusp edge portion which extends in a circumferential direction relative to the frame.

Example 3. The prosthetic heart valve of any example herein, particularly example 2, wherein the two angled side edges are coupled to the frame such that a material of the main body of the leaflet is compressed in the X direction and/or Y direction to form the concave shape

Example 4. The prosthetic heart valve of any example herein, particularly either example 2 or example 3, wherein the two angled side edges are compressed along their length to from the concave shape.

Example 5. The prosthetic heart valve of any example herein, particularly any one of examples 2-4, wherein the two angled side edges are compressed laterally toward one another to form the concave shape.

Example 6. The prosthetic heart valve of any example herein, particularly any one of examples 2-5, wherein the inflow edge is compressed along its length to form the concave shape.

Example 7. The prosthetic heart valve of any example herein, particularly any one of examples 2-6, wherein the inflow edge is parallel to straight portions of the outflow edge portion.

Example 8. The prosthetic heart valve of any example herein, particularly example 7, wherein the outflow edge portion comprises a central protrusion that extends axially outward from and is disposed between the straight portions of the outflow edge portion.

Example 9. The prosthetic heart valve of any one example herein, particularly any one of examples 2-8, wherein the inflow edge is coupled to the frame downstream of the inflow end of the frame.

Example 10. The prosthetic heart valve of any one example herein, particularly any one of examples 1-9, wherein the frame comprises a plurality of interconnected struts forming a plurality of circumferentially extending rows of angled struts arrayed along a length of the frame, between the inflow end and outflow end of the frame, wherein the inflow end of the frame comprises a plurality of inflow apices that are spaced circumferentially apart around the frame, and wherein an inflow end of the cusp edge portion of each leaflet is coupled to the frame downstream of the inflow apices.

Example 11. The prosthetic heart valve of any example herein, particularly example 10, wherein the plurality of inflow apices are formed by a first row of angled struts of the plurality of circumferentially extending rows of angled struts, wherein the frame comprises a plurality of non-apical junctions spaced circumferentially apart around the frame and connecting adjacent angled struts of the first row downstream of the inflow apices, and wherein the inflow end of the cusp edge portion of each leaflet is coupled to the frame between two non-apical junctions.

Example 12. The prosthetic heart valve of any example herein, particularly either example 10 or example 11, wherein the frame comprises a plurality of commissure supports spaced circumferentially apart around the frame, and wherein the cusp edge portion of each leaflet is coupled to a respective portion of angled struts of the plurality of circumferentially extending rows of angled struts that extend from one commissure support of the frame toward the inflow end of the frame, and from a location offset from the inflow end of the frame to an adjacent commissure support of the frame.

Example 13. The prosthetic heart valve of any example herein, particularly example 12, wherein each leaflet comprises opposing commissure tabs disposed on opposite sides of the main body, and wherein pairs of commissure tabs of adjacent leaflets are secured to respective commissure supports of the plurality of commissure supports.

Example 14. The prosthetic heart valve of any example herein, particularly any one of examples 1-13, further comprising an inner skirt disposed around an inside of the frame and attached to the frame with stitches, and wherein the cusp edge portion of each leaflet is attached to the inner skirt such that the cusp edge portion is coupled to the frame such that the leaflet is compressed in the X direction and/or Y direction to form the concave shape.

Example 15. A prosthetic heart valve comprising: a frame that is radially expandable from a radially collapsed configuration to a radially expanded configuration, wherein the frame has an inflow end and an outflow end; and a plurality of leaflets mounted on an inside the frame, wherein each leaflet comprises a main body with a cusp edge portion and an outflow edge portion, wherein the cusp edge portion has a trapezoidal shape with two angled side edges and an inflow edge extending between the two angled side edges, and wherein the inflow edge defines an inflow end of the cusp edge portion which extends in a circumferential direction relative to the frame, wherein the cusp edge portion is coupled to the frame such that a width of the leaflet measured from one side edge of the two angled side edges to the other side edge of the two angled side edges is smaller than a width of the leaflet prior to being coupled to the frame.

Example 16. The prosthetic heart valve of any example herein, particularly example 15, wherein the cusp edge portion is coupled to the frame such that the two angled side edges are compressed laterally toward one another, and the main body bulges outward to form a concave shape.

Example 17. The prosthetic heart valve of any example herein, particularly either example 15 or example 16, wherein the inflow edge is coupled to the frame downstream of the inflow end of the frame.

Example 18. The prosthetic heart valve of any example herein, particularly any one of examples 15-17, wherein the cusp edge portion is coupled to the frame such that the two angled side edges are compressed along their length.

Example 19. The prosthetic heart valve of any example herein, particularly any one of examples 15-18, wherein the inflow edge is coupled to the frame such that it is compressed along its length.

Example 20. The prosthetic heart valve of any example herein, particularly any one of examples 15-19, wherein the inflow edge is coupled to the frame such that a length of the inflow edge is shorter than a length of the inflow edge prior to being coupled to the frame.

Example 21. The prosthetic heart valve of any example herein, particularly any one of examples 15-20, wherein the inflow edge is straight.

Example 22. The prosthetic heart valve of any example herein, particularly any one of examples 15-21, wherein the inflow edge is parallel to straight portions of the outflow edge portion.

Example 23. The prosthetic heart valve of any example herein, particularly example 22, wherein the outflow edge portion comprises a central protrusion that extends axially outward from and is disposed between the straight portions of the outflow edge portion.

Example 24. The prosthetic heart valve of any example herein, particularly example 23, wherein the outflow edge portion comprises two depressed regions that depress inwards into the main body of the leaflet, and wherein the central protrusion is disposed between the two depressed regions.

Example 25. The prosthetic heart valve of any example herein, particularly any one of examples 15-24, wherein the frame comprises a plurality of interconnected struts forming a plurality of circumferentially extending rows of angled struts arrayed along a length of the frame, between the inflow end and outflow end of the frame.

Example 26. The prosthetic heart valve of any example herein, particularly example 25, wherein the inflow end of the frame comprises a plurality of inflow apices that are spaced circumferentially apart around the frame, and wherein the inflow edge of each leaflet is coupled to the frame downstream of the inflow apices.

Example 27. The prosthetic heart valve of any example herein, particularly example 26, wherein the plurality of inflow apices are formed by a first row of angled struts of the plurality of circumferentially extending rows of angled struts, wherein the frame comprises a plurality of non-apical junctions spaced circumferentially apart around the frame and connecting adjacent angled struts of the first row downstream of the inflow apices, and wherein the inflow edge of each leaflet is coupled to the frame between two non-apical junctions.

Example 28. The prosthetic heart valve of any example herein, particularly example 27, wherein a distance between the two non-apical junctions is smaller than a length of the inflow edge of the leaflet prior to being coupled to the frame.

Example 29. The prosthetic heart valve of any example herein, particularly either example 27 or example 28, wherein the frame comprises a plurality of commissure supports spaced circumferentially apart around the frame, and wherein the two angled side edges are coupled to a portion of angled struts of the plurality of circumferentially extending rows of angled struts that extend from a first commissure support of the frame to a first non-apical junction of the two non-apical junctions, and from a second non-apical junction of the two non-apical junctions to a second commissure support of the frame.

Example 30. The prosthetic heart valve of any example herein, particularly example 29, wherein each leaflet comprises opposing commissure tabs disposed on opposite sides of the main body, and wherein pairs of commissure tabs of adjacent leaflets are secured to respective commissure supports of the plurality of commissure supports.

Example 31. The prosthetic heart valve of any example herein, particularly example 30, wherein the opposing commissure tabs of each leaflet are angled inward toward the outflow edge portion prior to being coupled to the frame, and wherein when coupled to a respective commissure support, an outflow edge of each commissure tab straightens to be aligned with the outflow edge portion.

Example 32. The prosthetic heart valve of any example herein, particularly any one of examples 15-31, further comprising an inner skirt disposed around an inside of the frame and attached to the frame with stitches, and wherein the cusp edge portion of each leaflet is attached to the inner skirt.

Example 33. A prosthetic heart valve comprising: a frame that is radially expandable from a radially collapsed configuration to a radially expanded configuration, wherein the frame has an inflow end and an outflow end; and a plurality of leaflets mounted on an inside the frame, wherein each leaflet comprises a main body with a cusp edge portion and an outflow edge portion, wherein the cusp edge portion has a trapezoidal shape with two angled side edges and an inflow edge extending between the two angled side edges, and wherein the inflow edge defines an inflow end of the cusp edge portion which extends in a circumferential direction relative to the frame, wherein the cusp edge portion is coupled to the frame such that the leaflet is compressed axially and/or laterally to form a concave shape.

Example 34. The prosthetic heart valve of any example herein, particularly example 33, wherein the leaflet is compressed both axially and laterally to form the concave shape.

Example 35. The prosthetic heart valve of any example herein, particularly either example 33 or claim 34, wherein the inflow edge is coupled to the frame downstream of the inflow end of the frame such that the two angled side edges are compressed along their length and the leaflet is compressed axially to from the concave shape of the main body.

Example 36. The prosthetic heart valve of any example herein, particularly any one of examples 33-35, wherein the cusp edge portion is coupled to the frame such that the two angled side edges are compressed laterally toward one another to form the concave shape.

Example 37. The prosthetic heart valve of any example herein, particularly any one of examples 33-36, wherein the cusp edge portion is coupled to the frame such that the inflow edge is compressed along its length to form the concave shape.

Example 38. The prosthetic heart valve of any example herein, particularly any one of examples 33-37, wherein the inflow edge is parallel to straight portions of the outflow edge portion.

Example 39. The prosthetic heart valve of any example herein, particularly

example 38, wherein the outflow edge portion comprises a central protrusion that extends axially outward from and is disposed between the straight portions of the outflow edge portion.

Example 40. The prosthetic heart valve of any example herein, particularly example 39, wherein the outflow edge portion comprises two depressed regions that depress inwards into the main body of the leaflet, and wherein the central protrusion is disposed between the two depressed regions.

Example 41. The prosthetic heart valve of any example herein, particularly any one of examples 32-40, wherein the frame comprises a plurality of interconnected struts forming a plurality of circumferentially extending rows of angled struts arrayed along a length of the frame, between the inflow end and outflow end of the frame.

Example 42. The prosthetic heart valve of any example herein, particularly example 41, wherein the inflow end of the frame comprises a plurality of inflow apices that are spaced circumferentially apart around the frame, and wherein the inflow edge of each leaflet is coupled to the frame downstream of the inflow apices.

Example 43. The prosthetic heart valve of any example herein, particularly example 42, wherein the plurality of inflow apices are formed by a first row of angled struts of the plurality of circumferentially extending rows of angled struts, wherein the frame comprises a plurality of non-apical junctions spaced circumferentially apart around the frame and connecting adjacent angled struts of the first row downstream of the inflow apices, and wherein the inflow edge of each leaflet is coupled to the frame between two non-apical junctions.

Example 44. The prosthetic heart valve of any example herein, particularly any one of examples 41-43, wherein the frame comprises a plurality of commissure supports spaced circumferentially apart around the frame, and wherein the two angled side edges are coupled to a portion of angled struts of the plurality of circumferentially extending rows of angled struts that extend from a first commissure support of the frame to a location offset from the inflow end of the frame, and from the location offset from the inflow end of the frame to a second commissure support of the frame.

Example 45. The prosthetic heart valve of any example herein, particularly example 44, wherein each leaflet comprises opposing commissure tabs disposed on opposite sides of the main body, and wherein pairs of commissure tabs of adjacent leaflets are secured to respective commissure supports of the plurality of commissure supports.

Example 46. The prosthetic heart valve of any example herein, particularly any one of examples 32-45, further comprising an inner skirt disposed around an inside of the frame and attached to the frame with stitches, and wherein the cusp edge portion of each leaflet is attached to the inner skirt such that the leaflet is compressed axially and/or laterally to form the concave shape.

Example 47. A method of assembling a leaflet to a frame of a prosthetic valve, comprising: coupling the leaflet to the frame of the prosthetic valve, wherein the leaflet comprises a main body with a cusp edge portion and an outflow edge portion, wherein the cusp edge portion has two angled side edges and an inflow edge disposed between the two angled side edges and defining an inflow end of the cusp edge portion, and wherein the coupling the leaflet to the frame comprises: moving the two angled side edges closer together to decrease a width of the leaflet that is measured between the two angled side edges and cause outward bulging of the main body; and coupling the two angled side edges to the frame.

Example 48. The method of any example herein, particularly example 47, further comprising moving the inflow edge of the leaflet toward the cusp edge portion of the leaflet and coupling the inflow edge to the frame such that the inflow edge is offset away from inflow apices of the frame that define its inflow end.

Example 49. The method of any example herein, particularly example 48, wherein the moving the inflow edge of the leaflet toward the cusp edges portion and coupling the inflow edge such that it is offset away from the inflow apices of the frame causes the two angled side edges to be compressed along their length and causes further outward bulging of the main body of the leaflet.

Example 50. The method of any example herein, particularly any one of examples 47-49, further comprising compressing the inflow edge along its length and coupling the inflow edge to the frame such that the inflow edge is shortened.

Example 51. The method of any example herein, particularly example 50, wherein the frame comprises a plurality of interconnected struts forming a plurality of circumferentially extending rows of angled struts arrayed along a length of the frame, between the inflow end and outflow end of the frame, wherein a first row of angled struts of the plurality of circumferentially extending rows of angled struts form a plurality of inflow apices spaced circumferentially apart around the frame and a plurality of non-apical junctions spaced circumferentially apart around the frame, wherein each non-apical junction connects two adjacent angled struts of the first row of angled struts, downstream of the inflow apices, and wherein the inflow edge of the leaflet is coupled to the frame between two non-apical junctions.

Example 52. The method of any example herein, particularly any one of examples 47-51, wherein the leaflet comprises opposing commissure tabs that are disposed on opposite sides of the main body, wherein each commissure tab is angled toward the cusp edge portion of the leaflet such that an outer edge of the commissure tab is angled at a non-zero angle relative to a central longitudinal axis of the leaflet prior to coupling the commissure tab to the frame, and further comprising coupling each commissure tab to the frame and as a result of the coupling, moving each commissure tab away from the outflow edge portion such that it assumes a straightened configuration where the outer edge is closer to parallel with the central longitudinal axis.

Example 53. The method of any example herein, particularly any one of examples 47-51, wherein the leaflet comprises opposing commissure tabs that are disposed on opposite sides of the main body, wherein each commissure tab is angled toward the cusp edge portion of the leaflet such that an outflow edge of the commissure tab is angled at a non-perpendicular angle relative to a central longitudinal axis of the leaflet prior to coupling the commissure tab to the frame, and further comprising coupling each commissure tab to the frame and as a result of the coupling, moving each commissure tab away from the outflow edge portion such that its outflow edge is aligned with straight portions of the outflow edge portion of the leaflet that are disposed perpendicular to the central longitudinal axis.

Example 54. The method of any example herein, particularly any one of examples 47-53, wherein the cusp edge portion has a trapezoidal shape where the inflow edge extends between the two angled side edges in a circumferential direction relative to the frame.

Example 55. The method of any example herein, particularly example 54, wherein the inflow edge is parallel to straight portions of the outflow edge portion and perpendicular to a central longitudinal axis of the leaflet.

Example 56. The method of any example herein, particularly any one of examples 47-55, where the cusp edge portion of the leaflet comprises a plurality of pre-formed apertures spaced apart along the cusp edge portion, and wherein the coupling the leaflet to the frame comprises compressing the two angled side edges and the inflow edge along their respective lengths such that a spacing between adjacent apertures of the plurality of pre-formed apertures decreases and causes outward bulging of the main body of the leaflet.

Example 57. The method of any example herein, particularly any one of examples 47-56, wherein the coupling the leaflet to the frame comprises attaching the cusp edge portion of the leaflet to an inner skirt and attaching the inner skirt to struts of the frame on an inside of the frame.

Example 58. A leaflet for a prosthetic heart valve, comprising: a main body with a cusp edge portion and an outflow edge portion; and two tabs disposed on opposite sides of the main body, adjacent to the outflow edge portion, wherein the outflow edge portion comprises a central bump and two depressed regions that are depressed inward into the main body relative to the central bump, and wherein the central bump is disposed between the two depressed regions.

Example 59. The leaflet of any example herein, particularly example 58, wherein the two depressed regions are depressed inward into the main body relative to outflow edges of the two tabs.

Example 60. The leaflet of any example herein, particularly either example 58 or example 59, wherein outflow edges of the two tabs are continuous with the outflow edge portion.

Example 61. The leaflet of any example herein, particularly any one of examples 58-60, further comprising two tab bumps, each tab bump disposed between a respective tab and depressed region.

Example 62. The leaflet of any example herein, particularly any one of examples 58-61, wherein each depressed region is disposed adjacent to a respective tab of the two tabs.

Example 63. The leaflet of any example herein, particularly any one of examples 58-62, wherein the central bump extends axially outward from outflow edges of the tabs.

Example 64. The leaflet of any example herein, particularly any one of examples 58-62, wherein the central bump is axially aligned with outflow edges of the tabs.

Example 65. The leaflet of any example herein, particularly any one of examples 58-62, wherein a peak of the central bump is axially offset toward an inflow end of the cusp edge portion and away from outflow edges of the tabs.

Example 66. The leaflet of any example herein, particularly example 65, wherein the two tabs are angled away from one another such that the outflow edges of the two tabs are angled at a non-perpendicular angle relative to a central longitudinal axis of the leaflet.

Example 67. The leaflet of any example herein, particularly example 66, wherein outer edges of the two tabs are aligned with a cusp edge of the cusp edge portion.

Example 68. The leaflet of any example herein, particularly example 65, wherein the two tabs are angle toward one another such that the outflow edges of the two tabs are angled at a non-perpendicular angle relative to a central longitudinal axis of the leaflet.

Example 69. The leaflet of any example herein, particularly example 68, further comprising two tab bumps, each tab bump disposed between a respective tab and depressed region, and wherein the two tab bumps are configured to distance tension lines of the leaflet farther away from an outflow edge of the leaflet.

Example 70. The leaflet of any example herein, particularly any one of examples 58-69, wherein the cusp edge portion has a trapezoidal shape with two angled side edges and an inflow edge extending between the two angled side edges, and wherein the inflow edge defines an inflow end of the cusp edge portion.

Example 71. The leaflet of any example herein, particularly example 70, wherein the inflow edge is straight and disposed perpendicular to a central longitudinal axis of the leaflet.

Example 72. The leaflet of any example herein, particularly any one of example 58-69, wherein the cusp edge portion has circular shape with an angle of curvature of 120 degrees.

Example 73. The leaflet of any example herein, particularly any one of examples 58-69, wherein the cusp edge portion has a shape of a first cylinder intersecting with a second cylinder when attached to an annular frame of the prosthetic heart valve.

Example 74. The leaflet of any example herein, particularly any one of examples 58-73, wherein the cusp edge portion comprises a plurality of pre-formed apertures spaced apart along the cusp edge portion.

Example 75. The leaflet of any example herein, particularly any one of examples 58-74, wherein the leaflet comprises a polymeric material.

Example 76. The leaflet of any example herein, particularly any one of examples 58-74, wherein the leaflet comprises tissue.

Example 77. The leaflet of any example herein, particularly any one of examples 58-76, wherein the leaflet is configured to have a three-dimensional shape where the main body bulges outward relative to the cusp edge portion when the leaflet is coupled to an annular frame of the prosthetic heart valve.

Example 78. A three-dimensional leaflet comprising: a main body with a cusp edge portion and an outflow edge portion, wherein a portion of the cusp edge portion has a shape of a first cylinder intersecting with a second cylinder and the main body has a concave shape when attached to an annular frame of a prosthetic heart valve.

Example 79. The three-dimension leaflet of any example herein, particularly example 78, wherein the portion of the cusp edge portion has the shape of the first cylinder intersecting the second cylinder at a perpendicular angle.

Example 80. The three-dimension leaflet of any example herein, particularly example 78, wherein the portion of the cusp edge portion has the shape of the first cylinder intersecting the second cylinder at a non-perpendicular angle.

Example 81. The three-dimension leaflet of any example herein, particularly example 80, wherein the non-perpendicular angle is in a range of 1 to 30 degrees relative to a central longitudinal axis of the three-dimensional leaflet.

Example 82. The three-dimension leaflet of any example herein, particularly example 80, wherein the non-perpendicular angle is 10 degrees relative to a central longitudinal axis of the three-dimension leaflet.

Example 83. The three-dimension leaflet of any example herein, particularly any one of examples 78-82, wherein the portion of the cusp edge portion is an inflow end portion that is disposed across the main body from the outflow edge portion.

Example 84. The three-dimension leaflet of any example herein, particularly any one of examples 78-82, further comprising opposing commissure tabs disposed on opposite sides of the main body, and wherein the portion of the cusp edge portion is a majority of the cusp edge portion which extends between the opposing commissure tabs.

Example 85. The three-dimension leaflet of any example herein, particularly any one of examples 78-84, wherein an inflow end portion of the main body that is adjacent to the cusp edge portion is sphere-shaped when attached to the annular frame.

Example 86. The three-dimension leaflet of any example herein, particularly any one of examples 78-85, wherein the cusp edge portion has an angle of curvature in a range of 100 to 140 degrees.

Example 87. The three-dimension leaflet of any example herein, particularly any one of examples 78-85, wherein the cusp edge portion has a circular shape with an angle of curvature of 120 degrees.

Example 88. The three-dimension leaflet of any example herein, particularly any one of examples 78-87, further comprising opposing commissure tabs disposed on opposite sides of the main body, and wherein the commissure tabs are angled toward one another prior to being attached to the annular frame.

Example 89. The three-dimension leaflet of any example herein, particularly any one of examples 78-87, further comprising opposing commissure tabs disposed on opposite sides of the main body, and wherein the commissure tabs are angled away from one another prior to being attached to the annular frame.

Example 90. The three-dimension leaflet of any example herein, particularly any one of examples 78-89, wherein the outflow edge portion comprises a central protrusion that extends axially outward from and is disposed between straight portions of the outflow edge portion.

Example 91. The three-dimension leaflet of any example herein, particularly any one of examples 78-89, wherein the outflow edge portion comprises a central protrusion that extends axially outward from the main body of the leaflet and two depressed regions that depress inwards into the main body of the leaflet, and wherein the central protrusion is disposed between the two depressed regions.

Example 92. A method of forming a three-dimensional (3D) leaflet for a prosthetic valve, comprising: cutting and intersecting a first cylinder representing a leaflet with a second cylinder to form a 3D cut leaflet shape and flattening the 3D cut leaflet shape into a 2D cut leaflet shape to form a 2D leaflet cutting template; cutting a leaflet material in a flattened configuration with the 2D leaflet cutting template to form a cut, 3D leaflet with a rounded cusp edge; and coupling the 3D leaflet to an annular frame of the prosthetic valve such that it assumes a 3D configuration where a main body of the 3D leaflet has a concave shape.

Example 93. The method of any example herein, particularly example 92, wherein the cutting and intersecting the first cylinder with the second cylinder is performed virtually with computer aided design software.

Example 94. The method of any example herein, particularly either example 92 or example 93, wherein the first cylinder intersects with the second cylinder at a 90-degree angle.

Example 95. The method of any example herein, particularly either example 92 or example 93, wherein the first cylinder intersects with the second cylinder at a non-perpendicular angle.

Example 96. The method of any example herein, particularly example 95, wherein the non-perpendicular angle is between one and 30 degrees relative to a central longitudinal axis of the leaflet.

Example 97. The method of any example herein, particularly example 95, wherein the non-perpendicular angle is 10 degrees, and wherein the 3D leaflet has a parabolic cups edge.

Example 98. The method of any example herein, particularly any one of examples 92-97, wherein the cusp edge of the 3D leaflet has an angle of curvature in a range of 100 to 140 degrees.

Example 99. The method of any example herein, particularly any one of examples 92-98, wherein coupling the 3D leaflet to the annular frame comprises offsetting an inflow end of the cusp edge away from an inflow end of the annular frame.

Example 100. The method of any example herein, particularly any one of examples 92-99, wherein coupling the 3D leaflet to the annular frame includes coupling the cusp edge of the 3D leaflet to the annular frame such that it assumes the 3D configuration where the main body of the 3D leaflet has the concave shape that bulges outward relative to the cusp edge.

Example 101. A leaflet for a prosthetic heart valve, comprising: a main body with a cusp edge portion and an outflow edge portion, wherein the cusp edge portion has a trapezoidal shape with two angled side edges and an inflow edge extending between the two angled side edges, and wherein the inflow edge defines an inflow end of the cusp edge portion which extends perpendicular to a central longitudinal axis of the leaflet; and two tabs disposed on opposite sides of the main body, adjacent to the outflow edge portion, wherein the two tabs angle toward one another such that outer edges of the two tabs are angled at a non-zero angle relative to the central longitudinal axis of the leaflet.

Example 102. The leaflet of any example herein, particularly example 101, wherein the outflow edge portion comprises a central bump and two depressed regions that are depressed inward into the main body relative to the central bump, and wherein the central bump is disposed between the two depressed regions.

Example 103. The leaflet of any example herein, particularly example 102, further comprising two tab bumps, each tab bump disposed between a respective tab and depressed region, and wherein the two tab bumps are configured to distance tension lines of the leaflet farther away from an outflow edge of the leaflet.

Example 104. The leaflet of any example herein, particularly either example 102 or example 103, wherein the central bump is axially offset from outflow edges of the two tabs, toward the inflow edge.

Example 105. The leaflet of any example herein, particularly any one of examples 101-104, wherein the cusp edge portion comprises a plurality of pre-formed apertures spaced apart along the inflow edge and two angled side edges.

Example 106. A prosthetic heart valve comprising: a frame that is radially expandable from a radially collapsed configuration to a radially expanded configuration, wherein the frame has an inflow end and an outflow end; and a plurality of leaflets mounted on an inside the frame, wherein each leaflet comprises a main body with a cusp edge portion, an outflow edge portion, an inner main surface, and an outer main surface, wherein each leaflet is loaded along an axis generally parallel to the inner and outer main surfaces to form a concave shape.

Example 107. The prosthetic heart valve of any example herein, particularly example 106, wherein the cusp edge portion has a trapezoidal shape with two angled side edges and an inflow edge extending between the two angled side edges, and wherein the inflow edge defines an inflow end of the cusp edge portion which extends in a circumferential direction relative to the frame.

Example 108. The prosthetic heart valve of any example herein, particularly example 107, wherein the two angled side edges are coupled to the frame such that a material of the main body of the leaflet is loaded in the axis generally parallel to the inner and outer main surfaces to form the concave shape.

Example 109. The prosthetic heart valve of any example herein, particularly either example 107 or example 108, wherein the two angled side edges are compressed laterally toward one another to form the concave shape.

Example 110. The prosthetic heart valve of any example herein, particularly any one of examples 107-109, wherein the inflow edge is compressed along its length to form the concave shape.

Example 111. The prosthetic heart valve of any example herein, particularly any one of examples 107-110, wherein the inflow edge is coupled to the frame downstream of the inflow end of the frame.

Example 112. The prosthetic heart valve of any example herein, particularly any one of examples 106-111, further comprising an inner skirt disposed around an inside of the frame and attached to the frame with stitches, and wherein the cusp edge portion of each leaflet is attached to the inner skirt such that the cusp edge portion is coupled to the frame such that the leaflet is loaded along the axis generally parallel to the inner and outer main surfaces to form the concave shape.

Example 113. A method comprising sterilizing the prosthetic heart valve, leaflet, apparatus, and/or assembly of any example.

Example 114. A prosthetic heart valve of any one of examples 1-112, wherein the prosthetic heart valve is sterilized.

The features described herein with regard to any example can be combined with other features described in any one or more of the other examples, unless otherwise stated. For example, any one or more of the features of one leaflet can be combined with any one or more features of another leaflet. As another example, any one or more features of one prosthetic heart valve can be combined with any one or more features of another prosthetic heart valve.

In view of the many possible ways in which the principles of the disclosure may be applied, it should be recognized that the illustrated configurations depict examples of the disclosed technology and should not be taken as limiting the scope of the disclosure nor the claims. Rather, the scope of the claimed subject matter is defined by the following claims and their equivalents.

We claim:

1. A prosthetic heart valve comprising:
a frame that is radially expandable from a radially collapsed configuration to a radially expanded configuration, wherein the frame has an inflow end and an outflow end; and
a plurality of leaflets mounted on an inside the frame, wherein each leaflet comprises a main body with a cusp edge portion and an outflow edge portion,
wherein each leaflet is compressed in an X direction and/or Y direction to form a concave shape.

2. The prosthetic heart valve of claim 1, wherein the cusp edge portion has a trapezoidal shape with two angled side edges and an inflow edge extending between the two angled side edges, and wherein the inflow edge defines an inflow end of the cusp edge portion which extends in a circumferential direction relative to the frame.

3. The prosthetic heart valve of claim 2, wherein the two angled side edges are coupled to the frame such that a material of the main body of the leaflet is compressed in the X direction and/or Y direction to form the concave shape.

4. The prosthetic heart valve of claim 2, wherein the two angled side edges are compressed along their length to from the concave shape.

5. The prosthetic heart valve of claim 2, wherein the two angled side edges are compressed laterally toward one another to form the concave shape.

6. The prosthetic heart valve of claim 2, wherein the inflow edge is compressed along its length to form the concave shape.

7. The prosthetic heart valve of claim 2, wherein the inflow edge is parallel to straight portions of the outflow edge portion.

8. The prosthetic heart valve of claim 7, wherein the outflow edge portion comprises a central protrusion that extends axially outward from and is disposed between the straight portions of the outflow edge portion.

9. The prosthetic heart valve of claim 1, wherein the frame comprises a plurality of interconnected struts forming a plurality of circumferentially extending rows of angled struts arrayed along a length of the frame, between the inflow end and outflow end of the frame, wherein the inflow end of the frame comprises a plurality of inflow apices that are spaced circumferentially apart around the frame, and wherein an inflow end of the cusp edge portion of each leaflet is coupled to the frame downstream of the inflow apices.

10. A method of assembling a leaflet to a frame of a prosthetic valve, comprising:
coupling the leaflet to the frame of the prosthetic valve, wherein the leaflet comprises a main body with a cusp edge portion and an outflow edge portion, wherein the cusp edge portion has two angled side edges and an inflow edge disposed between the two angled side edges and defining an inflow end of the cusp edge portion, and wherein the coupling the leaflet to the frame comprises:
moving the two angled side edges closer together to decrease a width of the leaflet that is measured between the two angled side edges and cause outward bulging of the main body; and
coupling the two angled side edges to the frame.

11. The method of claim 10, further comprising moving the inflow edge of the leaflet toward the cusp edge portion of the leaflet and coupling the inflow edge to the frame such that the inflow edge is offset away from inflow apices of the frame that define its inflow end.

12. The method of claim 10, wherein the leaflet comprises opposing commissure tabs that are disposed on opposite sides of the main body, wherein each commissure tab is angled toward the cusp edge portion of the leaflet such that an outer edge of the commissure tab is angled at a non-zero angle relative to a central longitudinal axis of the leaflet prior to coupling the commissure tab to the frame, and further comprising coupling each commissure tab to the frame and as a result of the coupling, moving each commissure tab away from the outflow edge portion such that it assumes a straightened configuration where the outer edge is closer to parallel with the central longitudinal axis.

13. The method of claim 10, wherein the leaflet comprises opposing commissure tabs that are disposed on opposite sides of the main body, wherein each commissure tab is angled toward the cusp edge portion of the leaflet such that an outflow edge of the commissure tab is angled at a non-perpendicular angle relative to a central longitudinal axis of the leaflet prior to coupling the commissure tab to the frame, and further comprising coupling each commissure tab to the frame and as a result of the coupling, moving each commissure tab away from the outflow edge portion such that its outflow edge is aligned with straight portions of the outflow edge portion of the leaflet that are disposed perpendicular to the central longitudinal axis.

14. The method of claim 10, where the cusp edge portion of the leaflet comprises a plurality of pre-formed apertures spaced apart along the cusp edge portion, and wherein the coupling the leaflet to the frame comprises compressing the two angled side edges and the inflow edge along their respective lengths such that a spacing between adjacent apertures of the plurality of pre-formed apertures decreases and causes outward bulging of the main body of the leaflet.

15. A leaflet for a prosthetic heart valve, comprising:
a main body with a cusp edge portion and an outflow edge portion; and
two tabs disposed on opposite sides of the main body, adjacent to the outflow edge portion,
wherein the outflow edge portion comprises a central bump and two depressed regions that are depressed inward into the main body relative to the central bump, and wherein the central bump is disposed between the two depressed regions.

16. The leaflet of claim 15, wherein the two depressed regions are depressed inward into the main body relative to outflow edges of the two tabs.

17. The leaflet of claim 15, further comprising two tab bumps, each tab bump disposed between a respective tab and depressed region.

18. The leaflet of claim 15, wherein the two tabs are angled away from one another such that outflow edges of the two tabs are angled at a non-perpendicular angle relative to a central longitudinal axis of the leaflet.

19. The leaflet of claim 15, wherein the two tabs are angle toward one another such that outflow edges of the two tabs are angled at a non-perpendicular angle relative to a central longitudinal axis of the leaflet.

20. The leaflet of claim 15, wherein the cusp edge portion has circular shape with an angle of curvature of 120 degrees.
